(19) United States
(12) Patent Application Publication
(10) Pub. No.: US 2016/0340652 A1
(43) Pub. Date: Nov. 24, 2016

(54) CULTURE MEDIUM FOR STEM CELL DIFFERENTIATION

(71) Applicant: The USA, as represented by the Secretary, Dept. of Health and Human Services, Bethesda, MD (US)

(72) Inventors: Guokai Chen, Rockville, MD (US); Yongshun Lin, Gaithersburg, MD (US)

(21) Appl. No.: 15/160,650

(22) Filed: May 20, 2016

Related U.S. Application Data
(63) Continuation-in-part of application No. 15/023,221, filed on Mar. 18, 2016.
(60) Provisional application No. 61/879,840, filed on Sep. 19, 2013.

(51) Int. Cl. C12N 5/077

(52) U.S. Cl. CPC C12N 5/0657; C12N 2500/25; C12N 2500/36; C12N 2501/91; C12N 2501/999; C12N 2501/33; C12N 2506/02; C12N 2506/03; C12N 2506/45

(57) ABSTRACT

Media for the culture of pluripotent stem cells and differentiation of cardiomyocytes, and methods of using this media to produce cardiomyocytes. These media and methods can support cloning, differentiation and induced pluripotent stem (iPS) cell derivation.

### CROSS REFERENCE TO RELATED APPLICATION

This application is a continuation-in-part of U.S. patent application Ser. No. 15/023,221, filed Mar. 18, 2016, which is a national stage application under 35 U.S.C. 371 and claims the benefit of PCT Application No. PCT/US2014/056485 having an international filing date of Sep. 19, 2014, which designated the United States, which PCT application claimed the benefit of U.S. Provisional Application No. 61/879,840 filed Sep. 19, 2013. The disclosure of each of these applications is incorporated herein by reference.

### TECHNICAL FIELD

The invention relates to chemically defined culture media useful for cardiomyocyte derivation and methods of using these media.

### BACKGROUND

Human pluripotent stem cells (hPSCs), including human embryonic stem cells (hESCs) and human induced pluripotent stem cells (iPSCs), can be propagated indefinitely while still retaining the capacity to differentiate into all somatic cell types, and represent a potentially inexhaustible supply of human cells. E8 medium is a chemically-defined cell culture medium developed in the laboratory of Dr. James Thomson (University of Wisconsin-Madison), and is one of the most widely published/used feeder-free cell culture medium for hESCs and iPSCs. However, a problem remains that stem cells cannot survive at confluent or high density in low protein medium, such as E8, and high density culture is often used in large scale stem cell expansion and differentiation. The capacity to sustain survival at high density is critical for maintaining consistent stem cell cultures and avoiding the development of abnormal stem cells, and for proper stem cell differentiation.

Cardiovascular/heart disease is the number one cause of death. At the same time, the heart is often the main target of drug toxicity. Many cardiac treatments require organ transplantation or cell based therapies. In these therapies, it is advantageous to transplant healthy cardiac cells derived from the patient iPSCs. High quality cardiac cells are essential for excluding high-risk drugs. Patient-specific cardiac cells may also be used to test drug doses for personalized therapies. Thus, it is important to generate large quantities of patient-specific cardiac cells that could be used in therapy, diagnosis, and screening. Cardiomyocyte differentiation from ESC/iPSCs is usually conducted in high-density culture, and conventional procedures usually use albumin-containing, or other serum-product-containing, high protein culture conditions. Cardiomyocyte differentiation in chemically defined low-protein culture has not been previously realized.

### SUMMARY

The present invention is based, in part, on the surprising finding that addition of certain factors to a low protein culture media enhances pluripotent stem cell survival and differentiation efficiency. Some aspects of the present invention can be used to produce stem cells. Related aspects of the invention can be used to induce and/or otherwise improve differentiation of cardiomyocytes.

In certain aspects, this disclosure provides a low protein culture medium with defined chemical components that allows pluripotent stem cell maintenance and differentiation, particularly the differentiation of stem cells into cardiomyocytes. This includes the production of high quality cardiac cells from human embryonic and induced pluripotent stem cells in chemically defined conditions.

This disclosure provides cell culture media comprising DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; and, at least one compound selected from the group consisting of: CHIR99021, IWP-2, heparin, heparan, insulin, and Rho-associated coiled-coil containing protein kinase (ROCK) inhibitor Y-27632. These media may be albumin-free media. These media may also be substantially or completely free of insulin. These media may also be substantially or completely free of basic fibroblast growth factor (bFGF). These media may also be substantially or completely free of transforming growth factor beta (TGFβ).

These media may be formulated to contain a lipid mixture comprising arachidonic acid, cholesterol, linoleic acid, linolenic acid, myristic acid, oleic acid, palmitic acid, and palmitoleic acid. These media may be formulated to contain an antibiotic. The antibiotic may be selected from penicillin, streptomycin, and a combination of these antibiotics.

The at least one compound may consist of CHIR99021. Alternatively, or additionally, the at least one compound may consist of heparin. Alternatively, or additionally, the at least one compound may consist of heparin and IWP-2. Alternatively, or additionally, the at least one compound may consist of insulin.

This disclosure also provides in vitro methods of directed cardiomyocyte differentiation including culturing a stem cell to a confluence between about 70% and about 90% confluent, and then contacting the stem cells with a culture media of this disclosure, thereby inducing the differentiation of cardiomyocyte cells. These methods may include culturing the stem cell to a confluence of between 80% to 90% confluent. In these methods, the stem cell may be at least one of a totipotent, pluripotent, multipotent, oligopotent, or unipotent stem cell, an embryonic stem cell (ESCs), an induced pluripotent stem cell (iPSCs), a fetal stem cell, an adult stem cell, a human stem cell, a human embryonic stem cell, and a human induced pluripotent stem cell.

In an example embodiment, an in vitro method of directed cardiomyocyte differentiation is provided that includes culturing human induced pluripotent stem cells to a confluence between about 70% and about 90% confluent, and thereafter culturing the stem cells for about 24 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; an antibiotic; and CHIR99021, and thereafter culturing the stem cells for about 24 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; and an antibiotic, and thereafter culturing the stem cells for about 72 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; an antibiotic, heparin, and IWP-2, and thereafter culturing the stem cells for about 48 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; and an antibiotic. This exemplary amendment effectively induces the differentiation of cardiomyocyte cells.

This Summary is neither intended nor should it be construed as being representative of the full extent and scope of the present invention. Moreover, references made herein to “the present disclosure,” or aspects thereof, should be understood to mean certain embodiments of the present invention and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present invention will become more readily apparent from the Description of Embodiments, particularly when taken together with the drawings.

### DEFINITIONS

To facilitate an understanding of the present invention, a number of terms and phrases are defined below.

As used herein, the singular forms “a”, “an”, and “the” include plural forms unless the context clearly dictates otherwise. For example, reference to “a biomarker” includes reference to more than one biomarker.

Unless specifically stated or obvious from context, as used herein, the term “or” is understood to be inclusive.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “albumin-free medium” means that a medium does not contain albumin or an albumin replacement, or that it contains essentially no albumin or albumin replacement. For example, an “albumin-free medium” can contain less than about 10%, 9%, 8%, 7%, 6%, 5%, 4%, 3%, 2%, 1%, or 0.5% albumin, or an undetectable amount of albumin, wherein the culturing capacity of the medium is still observed.

As used herein, the term “cardiomyocyte” is meant to include cardiomyocyte progenitor cells having the ability to become functional cardiomyocytes in the future, as well as fetal and adult cardiomyocytes at all stages of differentiation. Cardiomyocytes can be identified by one or more than one marker or index.

As used herein, the term “cell culture objective” is meant to refer to any desired outcome of cell culture. Examples of culture objectives include, but are not limited to, cell survival, maintenance, passaging, proliferation, pluripotency, cloning, differentiation and induced pluripotent stem (iPS) cell derivation.

As used herein, the term “cloning” refers to initiating clonal colonies by growing human ES cell colonies from single individual ES cells, “cloning efficiency” refers to the number of individualized cells that form new cell colonies divided by the number of individualized cells plated in culture. Cloning efficiency varies considerably depending on culture conditions.

As used herein, the term “high density” is meant to refer to a high density of cells in culture. In certain embodiments, high density refers to growing stem cells at >70%, for example 70%, 75%, 80%, 85%, 90%, 95%. In certain embodiments, “high density” refers to >90% cell confluence for several days.

As used herein, “low protein medium” means that a medium contains a low percentage of protein. For example, a “low protein medium” can contain less than about 10%, 9%, 8%, 7%, 6%, 5%, 4%, 3%, 2% or 1% protein, wherein the culturing capacity of the medium is still observed. In example embodiments, the low protein medium is an albumin free medium.

As used herein, the term “passaging” is meant to refer to the process of dividing cells that have been cultivated in a culture vessel up to a certain density into aggregates, which are then placed into new culture vessels. These aggregates can contain any number of cells, typically between 100 to 1,000 cells, which readily initiate growth in culture.

As used herein, the term “pluripotent cell” means a cell capable of differentiating into cells of all three germ layers. Examples of pluripotent cells include embryonic stem cells and induced pluripotent stem (iPS) cells. As used herein, “iPS cells” refer to cells that are substantially genetically identical to their respective differentiated somatic cell of origin and display characteristics similar to higher potency cells, such as embryonic stem (ES) cells, as described herein. The cells can be obtained by reprogramming non-pluripotent (e.g. multipotent or somatic) cells.

As used herein, the term “pluripotent” or “pluripotency” is meant to refer to a cell's ability to differentiate into cells of all three germ layers. Pluripotent stem cells are meant to refer to cells which are capable of indefinite or long-term cell proliferation while remaining in an undifferentiated state in an in vitro culture, which retain normal karyotypes, and which have the ability to differentiate into all of three germ layers (ectoderm, mesoderm and endoderm) under appropriate conditions.

As used herein, the term “somatic cell reprogramming” is meant to refer to a process whereby somatic cells are reprogrammed to induced pluripotent stem cells (iPSCs).

The term “stem cell” is meant to refer to cells found in all multicellular organisms, that can divide (through mitosis) and differentiate into diverse specialized cell types and can self-renew to produce more stem cells. In mammals, there are two broad types of stem cells: embryonic stem cells, which are isolated from the inner cell mass of blastocysts, and adult stem cells, which are found in various tissues.

As used herein, the term “matrix” is meant to refer to a substrate that cells can be grown and cultured on. An exemplary matrix is MATRIGEL™.

As used herein, the term “volume expander” is meant to refer to a factor that is added to the culture medium that supports cell proliferation and differentiation. In example embodiments, the volume expander is a polysaccharide.

As used herein the term “lipid mix” is meant to refer to a chemically defined mixture of lipids for use in cell culture. An example lipid mix may comprise arachadonic acid, cholesterol, DL-alpha-tocopherol acetate, ethyl alcohol 100%, linoleic Acid, linolenic Acid, myristic acid, oleic acid, palmitic acid, palmitoleic acid, Pluronic F-68, stearic acid, tween-80. Another example lipid mix may comprise arachadonic acid (2 mg/ml), cholesterol (220 mg/ml), DL-alpha-tocopherol acetate (70 mg/ml), ethyl alcohol 100%, linoleic Acid (10 mg/ml), linolenic acid (10 mg/ml), myristic acid (10 mg/ml), oleic acid 10 mg/ml), palmitic acid (10 mg/ml), palmitoleic acid (10 mg/ml), Pluronic F-68 (90000 mg/ml), stearic acid (10 mg/ml), tween-80 (2200 mg/ml). Another example lipid mix is commercially available as Chemically Defined Lipid Concentrate (Cat#11905031) from Life Technologies.

As used herein, the term “about” means +/−10% of the recited value. Use of “about” is contemplated in reference to all ranges and values recited herein. With specific respect to the use of “about” in reference to hours of incubation of cells (e.g., “about 48 hours” or “about 24 hours”) means +/−4 hours (e.g., “about 48 hours” means 44 to 52 hours).

As used herein, the term “growth factor modulator” is meant to refer to a factor that is added to the culture medium that supports cell proliferation and differentiation. In certain embodiments, the growth factor modulator is heparin, heparan sulfate or dextran sulfate.

### BRIEF DESCRIPTION OF DRAWINGS

The present invention will be better understood and features, aspects and advantages other than those set forth above will become apparent when consideration is given to the following detailed description thereof. Such detailed description makes reference to the following drawings, wherein:

FIG. 1 shows the results of screening assays with indicated additives (trehalose, CM-dextran, lipid mix, heparin, as indicated). Heparin:20120807 Did not last long. H1 hESCs were cultured in E8 media to 60% confluence, and then continually cultured for 2 days in E8 media with indiated additives. Alkaline Phosphatase (AP) staining was performed to show the pluripotent stem cells. Dark staining represents the survival of hESC colonies.

FIG. 2 shows the results of screening assays with different additives (trehalose, CM-dextran, glycogen, albumin, lipid mix, heparin). H1 hESCs were cultured in E8 media to 60% confluence, and then continually cultured for 2 days in E8 media with indiated additives. Alkaline Phosphatase (AP) staining was performed to show the pluripotent stem cells. Dark staining represents the survival of hESC colonies.

FIG. 3 shows cell proliferation and differentiation under normal (“good incubator”) and suboptimal (“bad incubator”) conditions. Additives are indicated (glycogen, glycogen+lipid+heparin). H1 and H9 hESCs were cultured in E8 media to 60% confluence, and then continually cultured for 2 days in E8 media with indicated additives. Two parallel experiments were performed in incubators in either good or bad condition. Alkaline Phosphatase (AP) staining was used to show the pluripotent stem cells with Dark staining suggests the survival of hESC colonies. The results suggest that the indicated polymers help hPSCs survival in high density even in bad incubator environment.

FIG. 4 shows effects of different doses of heparin on over-confluent hESCs. H9 hESCs were cultured over-confluence for 3 days with/without different indicated doses of heparin, phase contrast imagers showed the survived cells.

FIG. 5 shows effects of different doses of additives (glycogen, DM-Dextran, and Trehalose) on over-confluent hESCs. H9 hESCs were cultured over-confluence for 3 days with/without different doses of polymers, phase contrast imagers showed the survived cells.

FIGS. 6a and 6b show cell proliferation and spontaneous differentiation at high density with trehalose and CM-dextran. In FIG. 6a, HI hESCs were cultured in E8 media to >90 confluence, and then continually cultured for 5 days in E8 media with indicated additives. Phase contrast images show the survival rates of the cells. Note that most of the cells in control conditions died and few cells regrew, while Trehalose and CM-Dextran treated cells survived in high confluence but some of them started to differentiate. In FIG. 6b, qPCR of pluripotent and differentiated genes expression is shown for the samples in FIG. 6a.

FIG. 7 shows a model of relationships between cell death, self-renewal, and differentiation, in low and high density culture. The top panels are shown at 5 days after confluence, at 10× magnification. The schematic in the bottom panel shows the relationship between cell death, self-renewal and differentiation at low density (left) and high density (right) culture.

FIG. 8 shows that the newly identified factors (indicated in the figure as CM-Dextran, glycogen, trehalose, heparin, or in some combinations) suppressed cell death during cardiomyocyte differentiation using the Test 1 protocol. Cell survival at day 3 is shown.

FIG. 9 shows that the newly identified factors (dextran, glycogen, trehalose, heparin, alone or in some combinations) suppressed cell death during cardiomyocyte differentiation at Test 3 condition. Cell survival at day 3 is shown.

FIG. 10 shows graphs that show the new factors that affected specific cardiac gene expression during differentiation. Expression of Nkx.2, TBX5 and cTnT are shown as determined by qPCR. qPCR was performed assess relative mRNA expression of cardiac markers for cells following Test3 protocol differentiation. Data showed that the indicated additives enhance cardiomyocyte differentiation.

FIGS. 11a and 11b show, respectively, a new protocol based on E8 conditions for cardiac production, and cardiac marker cTnT staining and FACS assay showed the efficiency of cardiomyocyte differentiation. The new protocol for cardiac differentiation was adapted from Lian X. et al., PNAS, 2012, using E8 basal media. Cardiac marker cTnT (green) staining and FAGS assay showed the efficiency of cardiomyocyte differentiation. Issues of E8 based cardiac differentiation protocol: 1. Lower than 50% derivation, 2. Inconsistent.

FIG. 12 shows that the new factors improved cardiomyocyte differentiation at new cardiac differentiation condition 1. H1 hESCs were differentiated in a new condition 1 protocol, as shown, and treated with the indicated factors (glycogen, heparin, glycogen+heparin and control), and the images of cells at day 18 are shown.

FIG. 13 shows that the new factors improved cardiomyocyte differentiation at new cardiac differentiation condition 2. H1 hESCs were differentiated in a new condition 2 protocol, as shown, and treated with the indicated factors (glycogen, heparin, glycogen+heparin and control), and the images of cells at day 18 are shown.

FIG. 14 shows the results of experiments screening for factors promoting cardiac differentiation. The indicated growth factors or inhibitors as shown on the x-axis were added at day 0-1 during cardiomyocyte differentiation. qPCR of cTnT expression suggested that Heparin significantly enhances cardiac derivation.

FIG. 15 is a graph that shows heparin is beneficial for cardiac differentiation with different WNT modulators as indicated (IWP2, IWR1, XAV939, KY). 1 μg/ml Heparin was added into media during cardiomyocyte differentiation using different WNT inhibitors, as indicated. FAC assay was assessed using cTnT antibody to show the positive rate of cardiomyocytes after 10 days differentiation. The results demonstrated that Heparin promoted cardiomyocyte production with all different WNT inhibitors.

FIG. 16 is a set of panels that show heparin promotes cardiomyocyte differentiation in the presence or absence of WNT inhibitors. Concentrations of WNT inhibitor (IWP2) and heparin are indicated, 1 μg/ml Heparin was added into media during cardiomyocyte differentiation with/without WNT inhibition. Immunofluorescence staining of cTnT antibody show the positive cardiomyocytes after 10 days differentiation. The results indicated that Heparin promoted cardiomyocyte production even without WNT inhibitors (nuclei were stained by DAPI).

FIG. 17 shows the results of microarray analysis that show heparin induced cardiac differentiation at different time points. mRNA was collected at indicated periods and submitted for microarray. The relative expression of cTnT from array data is shown.

FIG. 18 shows cardiomyocyte production with 1 μg/ml heparin treatment at different time periods. Time period is indicated below each panel. Timeline screening for heparin treatment was performed and the phase contrast images of 10 days differentiated cells are shown. The results suggested that treatment of Heparin from day 1 to day 7 helps cardiomyocyte production the most.

FIG. 19 shows the effects of different dosages of heparin (0.3-30 μg/ml) on cardiomyocyte differentiation. Dosage screening (0.3-30 μg/ml) for heparin treatment was performed and the immunofluorescence of cTnT (green) of 10 days differentiated cells is shown (nuclei were stained by DAPI, blue). The results suggested that treatment of 1 μg/ml Heparin helps cardiomyocyte production the most.

FIG. 20 shows a doxycycline (doxo) killing curve where cardiomyocytes were treated with doxo at indicated concentrations for two days. Cardiomyocytes derived from H9 hESCs and ND2 hiPSCs were treated at indicated dosages of Doxorubicin for two days, the cell viability was measured by PrestoBlue live cell viability kit.

FIG. 21 shows results of transplantation experiments. Cardiomyocytes carrying GFP derived from ND2 hiPSCs were injected into mouse surgery heart, the GFP labeled cells can be survived and detected in vivo several after injection. Cardiomyocytes carrying GFP derived from ND2 hiPSCs were injected into mouse surgery heart, the GFP labeled cells can survive and are detected in vivo several weeks after injection.

FIG. 22 shows results that demonstrated the general function of heparin on different human ESC/iPSC lines. Specifically, the effect of heparin on cardiac differentiation was tested on the multiple indicated human ESC and iPSC lines. The percentages of Troponin T (CTNT) positive cells at day 10 were detected by FACS. Ctrl: control; ip: treat PWP2 at day 2-5; h3: treat heparin (3 μg/ml) at day 1-7; ip, h3: treat PWP2 at day 2-5, and heparin (3 μg/ml) at day 1-7.

FIG. 23 is a schematic of an hPSC differentiation protocol with singular modulation of Wnt signaling in the absence of B27. The experiments were carried out in chemically defined E8 basal medium (DMEM/F12 plus L-ascorbic acid, Selenium, and Transferrin), with chemically defined lipid concentrate (lip).

FIG. 24 shows a screen of different signaling pathway regulators during cardiac specification (treated at day 2-5).

FIG. 25 shows the results of a heparin dosage screen when treated at day 2-5 in the presence of WNT inhibitor IWP2. CTNT positive cell percentage by flow cytometry at day 10 is shown.

FIG. 26 shows a real-time PCR of NKX2.5 showing the effect of a different time course of heparin (3 μg/ml) treatment in the presence of WNT inhibitor IWP2.

FIG. 27 shows a comparison of live cell number yield among different heparin dosage treatments after 10 days differentiation in the presence of IWP2 treatment.

FIG. 28 shows a comparison of live cell number yield after different time courses of heparin treatments after 10 days differentiation in the presence of IWP2 treatment.

FIG. 29 shows the results of FACS of cardiac marker CTNT indicating that the derived cardiomyocyte population was up to 95% pure under heparin and IWP2 treatment.

FIG. 30 shows electrophysiological characterization of the representative action potential activities of three cardiomyocyte subtypes at day 30 after differentiation (n=3 cells for atrial and ventricular types and n=2 cells for Nodal type).

FIG. 31 shows flow cytometry of CTNT (left) and real-time PCR of NKX2.5 (right) comparing the dosage (1-7 day treatment, left) and time course (3 μg/ml treatment, right) of heparin treatment to promote cardiac differentiation in the absence of IWP2 treatment.

FIG. 32 shows a comparison of live cell number yield among different doses of heparin treatment after 10 days differentiation in the absence of IWP2 treatment.

FIG. 33 shows how heparin upregulated WNT3A expression at mesoderm induction stage (day 2-3), but inhibited canonical WNT signaling downstream target, AXIN2, and downregulated cardiovascular progenitor cell marker, KDR, during cardiac specification stage (day 3-6).

FIG. 34 shows an early mesodermal and cardiovascular progenitor marker expression pattern during cardiac differentiation in the ND2 human iPSC cell line.

FIG. 35 shows that both IWP2 and heparin treatment inhibited Axing expression at day 5 in all 5 hPSC lines.

FIG. 36 shows a comparison of different Wnt inhibitors on cardiac differentiation with or without heparin treatment. The FACS results of cardiac troponin T (CTNT)-positive population after 10 days differentiation (left panel) and the quantified percentage of CTNT-positive cell population (right panel).

FIG. 37 shows the live cell number yield among different Wnt inhibitor treatments after 10 days differentiation in the absence (ctrl) or presence (hep) of heparin treatment.

FIG. 38 shows qPCR results from day 5 samples indicating that both IWP2 and heparin promoted cardiac progenitor marker GATA4.

FIG. 39 shows enriched function predictions by z score on microarray data indicating that heparin treatment decreased cell death, but increased cell growth and survival at day 3, while decreasing cell growth, but increasing cell differentiation at day 6.

FIG. 40 shows a model of the biochemical effects of heparin on biphasic regulation of canonical Wnt signaling during cardiac differentiation.

FIG. 41 shows a schematic of a hPSC differentiation protocol with modulation of Wnt signaling and treatment of heparin using a chemically-defined E8 medium platform.

FIG. 42 shows that heparin promoted the cardiac differentiation on all 8 hESC and hiPSC lines. Cardiac derivation efficiency at day 10 was shown by FACS with CTNT antibody.

FIG. 43 shows a comparison of the effects of different batches of heparin and recombinant human albumin on cardiac differentiation using the platform of E8 basal medium (left panel) or RPMI medium (right panel) in either the absence or presence of IWP2 treatment. Data shown is FACS using CTNT antibody. The batch numbers of the heparin or albumin batches used are listed.

FIG. 44 shows the effects of heparin and B27 (without insulin) on cardiomyocyte differentiation, compared on E8 basal medium and DMEM/F12 medium. Data shown is realtime PCR of cardiac troponin T (CTNT).

FIG. 45 shows the maturation of cardiomyocytes on culture plates. Immunofluorescence of α-ACTININ shows different myofibril content and sarcomere alignment between cardiomyocytes at 10 days and 50 days culture.

FIG. 46 shows the maturation of cardiomyocytes on culture plates. Immunofluorescence of CTNT (I) shows different myofibril content and sarcomere alignment between cardiomyocytes at 10 days and 50 days culture.

FIG. 47 shows the maturation of cardiomyocytes on culture plates. Immunofluorescence of MLC2a in CTNI positive cardiomyocytes indicates that MLC2a is expressed in the majority of early stage (10 days) but not late stage (50 days) cardiomyocytes.

FIG. 48 shows the maturation of cardiomyocytes on culture plates. Immunofluorescence of MLC2v in CTNT positive cardiomyocytes indicates that MLC2v is expressed in the majority of late stage (50 days) but not early stage (10 days) cardiomyocytes.

### DESCRIPTION OF EMBODIMENTS

Chemically defined culture medium has long been an ideal platform for stem cell research and applications. However, cell death, which occurs when cells reach high density, affects the maintenance and differentiation efficiency of stem cells. The treatments developed in undefined high protein media usually have to be adjusted or re-invented in defined low-protein conditions such as E8 media. It would be ideal to bridge the gap between the cells cultured under undefined high protein conditions and defined culture medium conditions. The present invention is based, in part, on the surprising finding that addition of certain factors to low protein culture medium enhances pluripotent stem cell survival and differentiation efficiency when the cells reach high density or when the conditions for differentiation require high density. The present invention provides low protein media that support maintenance, proliferation, or differentiation of pluripotent stem cells. These media may comprise one or more polysaccharides. The present invention further provides a method for cardiac differentiation from stem cells using heparin and heparan sulfate to supplement the low protein medium.

### Pluripotent Stem Cells

Pluripotent cells, such as embryonic stem (ES) cells and induced pluripotent stem (iPS) cells, have the potential to differentiate into cells of all three primary germ layers (Thomson, et al., Science 282, 1145-47 (1998)). Pluripotent stem cells exist only transiently during embryogenesis, and the cells in culture are an artifact of cell culture conditions. The remarkable developmental potential of pluripotent cells has proven useful for basic research and clinical application. Many basic methods for human pluripotent cell culture, such as growth media, plate coating, and other conditions, have been developed and refined (Ludwig et al., Nat. Biotechnol 24, 185-87 (2006); Ludwig et al., Nat. Methods 3, 637-46 (2006)). For example, while human ES cells were initially cultured in fetal bovine serum (FBS)-containing media on murine embryonic fibroblast (MEF) feeder cells, fully defined media as well as defined protein matrices are now available (Ludwig et al, Nat. Biotechnol 24, 185-187 (2006)). Pluripotent cell culture methods have evolved considerably. Several growth media were developed that provide basic nutrients and growth factors for survival and expansion of pluripotent cells and directly determine how cells grow and differentiate. TeSR was one of the first defined media that supports pluripotent cell maintenance in an undifferentiated state in the absence of feeder cells or conditioned medium through multiple culture passages (Ludwig et al., Nat. Methods 3, 637-46 (2006); U.S. Pat. No. 7,449,334, each of which is incorporated herein by reference in its entirety). TeSR contains 18 components in addition to the basal medium DMEM/F12 that itself has 52 components. A complete list of ingredients of DMEM/F12 is set forth in Table 1.

TABLE 1

| Inorganic Salts (g/liter) | Amino Acids (g/liter) | Vitamins (g/liter) | Other (g/liter) |
|---|---|---|---|
| CaCl2 (anhydrous) | L-Al [symbol] 0.0044 [symbol] | D- [symbol] 0. [symbol] 365 | D-Glucose 3.15100 |
| 0.1166 [symbol] | L-A [symbol] HCl 0.14750 | Ch [symbol] Chloride 0.00 [symbol] | HEPES 3.574 [symbol] 0 |
| C [symbol] SO4 (anhydrous) | L-Asparagine H2O | Folic Acid 0.0026 [symbol] | H [symbol] 0.00239 |
| 0.0000008 | 0.007 [symbol] 0 | [symbol] [symbol] 0.01261 | Linoleic Acid 0.000044 |
| F [symbol] (NO3)3•9H2O 0.0000 [symbol] | L-A [symbol] Acid 0.006 [symbol] | Niacinamide 0.002 [symbol] 2 | Phenol Red Sodium [symbol] |
| FeSO4•7H2O 0.000417 | L-Cystine-HCl•H2O | D-P [symbol] Acid | 0.00 [symbol] |
| MgSO4 (anhydrous) | 0.017 [symbol] 6 | 0.00224 | P [symbol] 2HCl 0.000 [symbol] |
| 0.0849 [symbol] | L-Cystine 2HCl 0.03129 | Pyrid [symbol] HCl 0.00203 | Pyruvic Acid Na 0.0 [symbol] 00 |
| KCl 0.3118 | L-Glutamic Acid 0.0073 [symbol] | Riboflavin 0.00022 | DL [symbol] Th [symbol] Acid |
| NaHCO3 1.20000 | L-Glutam 0.36 [symbol] 10 | Thiamine HCl 0.00217 | 0.000105 |
| NaCl 7.00000 | Glycine 0.01875 | Vitamin B-12 0.00668 | Thymidine 0.000 [symbol] |
| Na2 [symbol] O4 (anhydrous) | L-H [symbol] HCl•H2O | | |
| 0.07100 | 0.03148 | | |
| N [symbol] 2PO4•H2O 0.0 [symbol] 2 [symbol] | L-Isoleucine 0. [symbol] 437 | | |
| ZnSO4•7H2O 0.0 [symbol] 432 | L-Leucine 0.0 [symbol] 89 [symbol] | | |
| | L-Lysine HCl 0.0 [symbol] 13 [symbol] | | |
| | L- Methionine 0.01724 | | |
| | L-P [symbol] 0.0 [symbol] 48 | | |
| | L-Proline 0.0 [symbol] 25 | | |
| | L-Serine 0.02625 | | |
| | L-Threoninr 0.0 [symbol] 3 [symbol] | | |
| | L-Tryptophan 0.0 [symbol] 902 | | |
| | L-Tyrosine [symbol] 2Na•2H2O | | |
| | 0.05582 | | |
| | L-Valine 0.0 [symbol] | | |

[symbol]

indicates data missing or illegible when filed

Although TeSR could be used to derive human ESCs in the complete absence of animal proteins, the inclusion of human serum albumin and human-sourced matrix proteins makes those conditions prohibitively expensive, impractical for routine use, are inconsistent from batch to batch, and not truly completely defined.

To fully exploit the potential of pluripotent cells for drug discovery, testing, and transplantation therapy, derivation and growth of these cells under fully-defined and, ideally, xeno-free, conditions is desirable.

PCT patent application Publication No. WO/2012/019122, incorporated by reference in its entirety herein, describes E8 culture medium as a fully-defined medium for pluripotent cells. E8 medium is a chemically defined stem cell medium that is the most widely published feeder-free cell culture medium for human ES cells and iPS cells, with established protocols for applications ranging from derivation to differentiation. WO/2012/019122 and Chen et al. (Nature Methods. 2011 Apr. 10; 8(4):424-429), describe the E8 medium. E8 medium is completely defined by eight components: DMEM/F12 (ingredients listed above in Table 1), L-ascorbic acid, selenium, transferrin, NaCHO₃, insulin, FGF2, and TGF-beta or nodal.

E8 medium is not able to support cell growth at higher density, and it is difficult to maintain or differentiate cells at confluent density. Among the phenotypes related to high density culture with E8, are that most cells die after 1-2 days of over-confluent culture, and the surviving cells are often abnormal.

High density cell culture is important for a number of reasons, including: consistency of maintenance; to avoid the rise of abnormal stem cells; stem cells need high density to properly differentiate; and, most current protocols use high density culture. Previous attempts to grow cells at high density have included the following supplements to the culture medium: small molecules (Rho-associated, coiled-coil containing protein kinase (ROCK) inhibitors, Caspase inhibitors), lipid carriers (different cyclodextrins), albumin peptides, polymers (Polyvinylpyrrolidone, Poly(vinyl alcohol)), growth factors (different bioactive proteins), nutrients (vitamins, energy source), albumin, starches, alginate, oxygen level, CDM, Chemically defined medium for high density cell culture. None of these approaches have been successful.

The present invention takes a new direction in providing media for the culture of stem cells. The stem cells may be totipotent, pluripotent, multipotent, oligopotent or unipotent stem cells. The stem cells may be embryonic stem cells, induced pluripotent stem cells, fetal stem cells, or adult stem cells. In example embodiments, the stem cell is a mammalian stem cell, for example a human stem cell, and more particularly a human embryonic stem cell or a human induced pluripotent stem cell.

The inventors have surprising found that the addition of certain factors to low protein culture medium enhances pluripotent stem cell survival and differentiation efficiency.

Low protein medium refers to a medium that has a low percentage of protein. For example, a “low protein medium” can contain less than about 10%, 9%, 8%, 7%, 6%, 5%, 4%, 3%, 2% or 1% protein, wherein the culturing capacity of the medium is still observed. In example embodiments, the low protein medium is an albumin-free medium. In further exemplary embodiments, the low protein medium is an E8 basal medium (consisting of E8 media without NaHCO3, insulin, basic fibroblast growth factor (also known as bFGF, FGF2 or FGF-β) or transforming growth factor beta (TGFβ); i.e., containing DMEM/F-12, L-ascorbic acid, selenium, and transferrin).

Factors that have been added to the low protein medium to support pluripotent stem cell survival and differentiation include a combination of factors, as set forth below. These factors may include factors for growth factor control, including for example, but not limited to, heparin and heparan sulfate. These factors may include a membrane stabilizer or volume expander, such as, but not limited to, trehalose, CM-Dextran, polysucrose, and/or glycogen.

Factors may also be added to supply cellular membrane components and essential lipids, and optionally include a mixture of lipids. In example embodiments, the lipid mix comprises arachadonic acid, cholesterol, DL-alpha-tocopherol acetate, ethyl alcohol 100%, linoleic acid, linolenic acid, myristic acid, oleic acid, palmitic acid, palmitoleic acid, Pluronic F-68, stearic acid, tween-80. In other example embodiments, the lipid mix comprises arachadonic acid (2 mg/ml), cholesterol (220 mg/ml), DL-alpha-tocopherol acetate (70 mg/ml), ethyl alcohol 100%, linoleic acid (10 mg/ml), linolenic acid (10 mg/ml), myristic acid (10 mg/ml), oleic acid 10 mg/ml), palmitic acid (10 mg/ml), palmitoleic acid (10 mg/ml), Pluronic F-68 (90000 mg/ml), stearic acid (10 mg/ml), tween-80 (2200 mg/ml). In further example embodiments, the lipid mixture is commercially available as Chemically Defined Lipid Concentrate (Cat#11905031) from Life Technologies.

In example embodiments, effective concentrations of specific factors are as follows: Trehalose, CM-Dextran, Glycogen, beta-cyclodextrin, beta-hydrodextrin, N-Acetyl-Glucosamine, Methyl alpha-D-Glycogyrate, Methyl beta-D-glycogyrate, Dextrin 100K, and Methyl-1-beta-cyclodextrin, are optionally used at concentrations ranging from 0.5-40 mg/ml. In certain embodiments these factors are used at concentrations ranging from 2-20 mg/ml, for example 2, 5, 10, and 20 mg/ml. In certain embodiments, Trehalose, CM-Dextran, Glycogen, Albumin, beta-cyclodextrin, beta-hydro dextrin, N-Acetyl-Glucosamine, Methyl alpha-D-Glycogyrate, Methyl beta-D-glycogyrate, Dextrin 100K, Methyl-1-beta-cyclodextrin, are optionally used at concentrations of 5 mg/ml, and/or 10 mg/ml.

Polysucrose is optionally used at a concentration of 4-20 mg/ml, for example 5, 10, 15 and 20 mg/ml. In certain embodiments, polysucrose is used at a concentration of 10 mg/ml.

Glycogen is optionally used at a concentration of 1-20 mg/ml, for example 1.0, 2.0, 2.5, 5.0, 10 and 20 mg/ml. In certain embodiments, glycogen is used at concentrations of 2.5 mg/ml and 5 mg/ml.

Heparin is used at concentrations between about 0.3-20 μg/ml, for example 0.5, 1.0, 2.0, 5.0, 10, and 20 μg/ml when cells are fed at confluent density. In certain embodiments, heparin is used at a concentration of about 20 μg/ml. In other embodiments, heparin is used at a concentration of about 2 μg/ml when cells are fed one day before reaching confluency.

In example embodiments, factors may also be removed from the low protein medium. For example, in certain embodiments, growth factors are removed from the medium. In particular, TGF, FGF and insulin may be removed from the low protein medium. Thus, media of this invention may be substantially, or completely, free of one or more growth factors selected from TGF, FGF, and insulin.

Heparin-containing or other related low protein medium described herein can be applied to cells at any of days −2, −1, 0, 1, 2, 3, 4, 5, 6, 7, 8, 9 and/or 10, with a regularity of once a day, twice a day, three times a day, four times a day, etc., e.g., hourly, qid, tid, bid, or qd. Similarly, application of such low protein medium may be withdrawn or halted or refreshed or replaced at any of days −1, 0, 1, 2, 3, 4, 5, 6, 7, 8, 9, 10, 11 and/or 12 or later.

Accordingly, this disclosure provides media useful for the culture of stem cells. In example embodiments, the media comprise one or more polysaccharides. In example embodiments, the media is a low protein medium, which may be an albumin-free medium.

In these media, polysaccharides may be included that are selected from the group consisting of trehalose, CM-Dextran or glycogen.

These media may comprise heparin, heparan sulfate, or dextran sulfate.

These media may comprise a lipid mixture.

These media support cell culture objectives, such as, but not limited to cell survival, maintenance, passaging, proliferation, pluripotency, cloning, differentiation, and induced pluripotent stem (iPS) cell derivation.

In one aspect, this disclosure provides methods for deriving cardiac cells under defined conditions that include culturing a pluripotent stem cell in a low protein medium described herein.

The expression of various markers specific to cardiomyocytes is detected by conventional biochemical or immunochemical methods. For example, an immunochemical method such as immunohistochemical staining or Immunoelectrophoresis may be used. In these methods, marker-specific polyclonal antibodies or monoclonal antibodies can be used which react with cardiomyocyte progenitor cells or cardiomyocytes. Antibodies for individual specific markers are commercially available, and can be easily used. Markers specific to cardiomyocyte progenitor cells or cardiomyocytes may include, but are not limited to, TBX5, TNNT2, NKX2-5, myosin heavy and light chains, a-actinin, troponin 1, ANP, GATA-4, MEF-2c, and the like.

Alternatively, expression of cardiomyocyte progenitor cell-specific or cardiomyocyte-specific marker genes can also be confirmed by molecular biological methods, such as microarray, reverse transcriptase polymerase chain reaction (RT-PCR) and hybridization analysis, which have been commonly used in the past for amplifying, detecting and analyzing mRNA encoding any marker proteins. The nucleic acid sequences encoding marker proteins specific to cardiomyocyte progenitor cells and cardiomyocytes (such as TBX5, TNNT2, NKX2.5) are already known and are available through public databases such as GenBank, and the marker-specific sequences needed for use as primers or probes can be easily determined.

Physiological indexes can also be used to confirm differentiation of ES cells into cardiomyocytes. For example, useful markers include the ability of cells derived from ES cells to beat spontaneously, and the ability of cells derived from ES cells to react to electrophysiological stimulus through various ion channels expressed on the cells.

Any method suited to inducing differentiation of cardiomyocytes can be used as the culture method for preparing cardiomyocytes from hPSC (human pluripotent stem cells, ESC/iPSC) cells in the present invention. For example, cell culture can be in adherent culture, suspension culture, soft agar culture, micro-carrier culture, and the like.

Cardiomyocytes prepared according to the present invention can be used in therapies to induce myocardial regeneration, or in therapies to treat heart disease in a mammal. Examples of heart disease that can be treated with cardiomyocytes prepared according to the methods of the present invention include myocardial infarction, ischemic heart disease, congestive heart failure, hypertrophic cardiomyopathy, dilative cardiomyopathy, myocarditis, chronic heart failure, and the like. When used to in therapies for myocardial regeneration or heart disease, cardiomyocytes prepared according to the present invention can be included in any form as long as the purity is high, such as cells suspended in the medium or other aqueous carrier, cells embedded in a biodegradable substrate or other support, or cells made into a single-layer or multilayer myocardial sheet.

Although not particularly limited to these, methods for transporting the cardiomyocytes prepared according to the present invention to a site in the body of a patient being treated include direct injection into the heart via an open chest and a syringe, transplantation via a surgical incision in the heart, and infusion via blood vessels using a catheter, all of which have been described in the art (Murry et al., Cold Spring Harb. Symp. Quant. Biol. 67:519, 2002; Menasche, Ann. Thorac. Surg. 75:S20, 2003; Dowell et al., Cardiovasc. Res. 58:336, 2003). Good therapeutic effects have been reported when cardiomyocytes collected from a fetal heart were transplanted by such methods to the hearts of animals with heart damage (Menasche, Ann. Thorac. Surg. 75:S20, 2003; Reffelmann et al., Heart Fail. Rev. 8:201, 2003). Cardiomyocytes derived from ES cells have characteristics similar to those of cardiomyocytes derived from fetal hearts (Maltsev et al., Mech. Dev. 44:41, 1993; Circ. Res. 75:233, 1994). Further, a high acceptance rate, equivalent to that achieved with fetal myocardial transplantation, has been confirmed in animal experiments in which cardiomyocytes derived from ES cells were actually transplanted into adult hearts (King et al., J. Clin. Invest. 98:216, 1996). Accordingly, it is expected that supplementary transplantation of cardiomyocytes prepared according to the present invention into diseased heart tissue should stimulate improved heart functions.

Each publication or patent cited herein is incorporated herein by reference in its entirety. The disclosure now being generally described will be more readily understood by reference to the following examples.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Deriving Human Pluripotent Stem Cells in E8 Based Growth Conditions

Human pluripotent stem cells (hPSCs), including human embryonic stem cells (hESCs) and human induced pluripotent stem cells (hiPSCs), have the potential to become the source materials for cell-based therapy, so the quality of the stem cells has great impact on how the cells could be utilized in future applications. Derivation and maintenance conditions have critical role determining the iPSC quality, mainly due to the involvement of animal products and feeder cells. A procedure was developed to derive and maintain hiPSCs in chemically defined media.

The following sets forth how to derive hiPSCs in E8 based growth conditions. This method has been successfully used on fibroblast, pre-adipocyte and HUVEC. The procedure can be adapted to most common reprogramming methods, such as Lentivirus, Episomal DNA and Sendai Virus. In comparison with E8 medium and vitronectin (or a synthetic surface), human pluripotent stem cells are preferably maintained in an enzyme-free, xeno-fee, and chemically defined environment. To simplify the description, Sendai Virus is used to reprogram fibroblasts, and MATRIGEL™ is used as the coating surface for cell culture in this exemplary protocol. However, the invention is not limited to either.

Plate coating: First (1), pour cold 12 ml of DMEM/F12 in conical tube, and use 1.5 ml to resuspend 2 mg frozen MATRIGEL™ with 5 ml pipet. MATRIGEL™ should be in freezer right before the experiment. Next, (2) rinse the MATRIGEL™ tube again with the same media. (3) Mix the MATRIGEL™ well, plate 1 ml in each well of 6-well), and shake well to cover all the surface. (4) Leave the plate at room temperature or 37° C. for at least 30 minutes. Or coat overnight at 4° C. EDTA dissociation buffer: for iPSCs (1) Add 500 ul 0.5M EDTA and 0.9 g NaCl into 500 ml Calcium/Magnesium free PBS (Invitrogen #14190). (2) Sterilize by filtration, and store at 4° C. Reprogramming with Sendai Virus.
1—Day 0—Take low passage # fibroblast culture and plate in 1 well of a 6 well dish so that cells will be about 80% confluent next day (100K or 150K cells/well) in fibroblast Media (usually 10% FBS and Pen/Strep, L-glut, NEAA in DMEM).
2—Day 1—Thaw the four Sendai Viruses on ice, mix together, and drop-wisely add them to the cells. Incubate at 37° C.
3—Day 2—If cells look good in the morning and nearly confluent, prepare plates for splitting. Coat 6 well plates with MATRIGEL™.
4—Still Day 2—Pass reprogramming well using TryPLE (Note, some lines do not come off plate well with TryPLE, with those lines, it may be advantageous to wash plate 2× with EDTA before adding TryPLE). Incubate 5 min in incubator, then wash off plate and dilute with fibroblast media.
5—Spin cells down and resuspend in fibroblast media. Plate on MATRIGEL™ (1 well of infected cells into 2×6 well plates coated with MATRIGEL™), also in Reprogramming Medium I.
6—Keep cells in Reprogramming Medium I, feeding them every other day for 3->5 days.
7—Day 5 or 7 (approximately)-change media to Reprogramming Medium II, and 100 RIVI Sodium Butyrate can be added to improve reprogramming efficiency.
8—Continue feeding every other day.
9—Depending on cell density and the appearance of any iPS colonies in the reprogramming plates, cells may need to be passed with EDTA at some point in the next 2 weeks. Also, it may be necessary to start feeding E8 medium without sodium butyrate.
10—Around Day 20-25, colonies should be ready to try picking. Around this time original reprogramming plates should begin being fed normal E8 media (with TGF I) daily, if they haven't been switched to this already.
11—For picking—prep a 24 well plate by coating with MATRIGEL™. After coating, add E8 (TGF I media) with 1× Rock inhibitor added to each well. Spray microscope and surrounding area as well as pipet and box of tips with alcohol. Wear face mask. Find colonies under scope with 4× objective. Using P20 with tip, circle around colony until it is loosened from surrounding cells. Also using tips, cross hatch the colony so it will come off plate in smaller pieces. Then use pipet to push colony off plate and suck it into pipette. Transfer colony pieces into 1 well on the 24 well plate. Repeat with other colonies. The day after picking, change media to E8 (TGFbI) without rock inhibitor. Feed daily until colony is big enough to pass.
12—When colony is ready to pass, use EDTA to pass and leave some of the cells in the original well of the 24 well while transferring most to a new MATRIGEL™ coated well on a 12 or 6 well plate, (in Rock inhibitor).
13—When expanding each colony, keep track of passage number (picking into 24 well is passage 1). Once you have a well in a 6 well ready to pass, freeze 2 vials down and leave some cells in the well to continue to grow them.
14—The usual order for cells is expand and freeze 2 vials from first well. Expand well on next passage to 2-3 wells. Freeze 4-6 vials when ready, and continue growing cells. Freeze at least 6 vial, up to 10 for each clone. During one passage, pass a small amount onto 12 well plate for APS staining. After getting enough cells for freezing, continue growing to harvest some for FACS staining.

### Materials

Cell Materials: Human Fibroblasts, Human iPSCs
Cell Culture Media: Fibroblast medium: 10% Fetal Bovine Serum in DMEM, 1× Non-essential amino acid.
Basic chemically defined reprogramming medium I: DMEM/F12, 64 mg/L-Ascorbic acid 2-phosphate magnesium salt, 14 pg/L Sodium Selenite, 10.7 mg/L Holo-transferrin, 100 μg/L basic FGF, 20 mg/L Insulin, and 1 pM Hydrocortisone. Adjust to pH7.4 with 340 mOsm osmolality. Basic chemically defined reprogramming medium II: DMEM/F12, 64 mg/L L-Ascorbic acid 2-phosphate magnesium salt, 14 μg/L Sodium Selenite, 10.7 mg/L Holo-transferrin 10.7 mg/L, 100 μg/L basic FGF, and 20 mg/L Insulin. Adjust to pH7.4 with 340 mOsm osmolality. (100 uM Sodium Butyrate can be added to improve reprogramming efficiency.)
Chemically defined human ESC/iPSC E8 medium: DMEM/F12, 64 mg/L L-Ascorbic acid 2-phosphate magnesium salt, 14 μg/L Sodium Selenite, 10.7 mg/L Holo-transferrin 10.7 mg/L, 100 pg/L basic FGF, 1.8 pg/L TGFB 1, 20 mg/L insulin. Adjust to pH7.4 with 340 mOsm osmolality.

### EDTA PBS (1000 ml)

| Ingredient | Amount | | Company | Catalog# |
|---|---|---|---|---|
| PBS | 500 | ml | Life Technology | 14190-250 |
| [symbol] 5M EDTA | 0.5 | ml | K.D. Biomedical | RGF3130 |
| NACl | 0.9 | g | Sigma | 5886 |

[symbol]

indicates data missing or illegible when filed

### Medium Reagents

| Ingredient | Company | Catalog# |
|---|---|---|
| DMEM/F12 | Life Technology | 11330 |
| L-Ascorbic acid 2-phosphate magnesium salt | Sigma | A8960 |
| Sodium Selenite | Sigma | S5261 |
| Sodium Chloride | Sigma | S5886 |
| Holotransferrin | Sigma | T066 |
| Basic FGF | eprotech | 100-18B |
| TGFB 1 | R&D Systems | 240-B/CF |
| Insulin | Sigma | 19278 |
| Hydrocortisone | Sigma | H0396 |
| Sodium Butyrate | Sigma | B5587 |

Materials: Inverted microscope (i.e., Nikon TE or Olympus ΓX or Zeiss Promo Vert); Biosafety cabinet for cell culture; CO₂ incubator with controlling and monitoring system for CO₂, humidity and temperature; Cell culture disposables: Tissue culture dishes, centrifuge tubes, pipettes, pipette tips, cell strainer etc.

Troubleshooting for the below issues is described, although not limited to the following:

Xeno-free condition for iPSC derivation: MATRIGEL™ may be replaced with Vitronectin recombinant protein. PBS-Containing fibroblast medium could be replaced with defined fibroblast medium.
Low reprogramming efficiency: Use sodium butyrate to improve the efficiency.

Initial fibroblasts are not actively proliferating or at high passage: Increase the experiment scale, and use more starting cells and viruses.

### Example 2

### Identification of New Factors that Enhance Pluripotent Stem Cell Survival and Differentiation

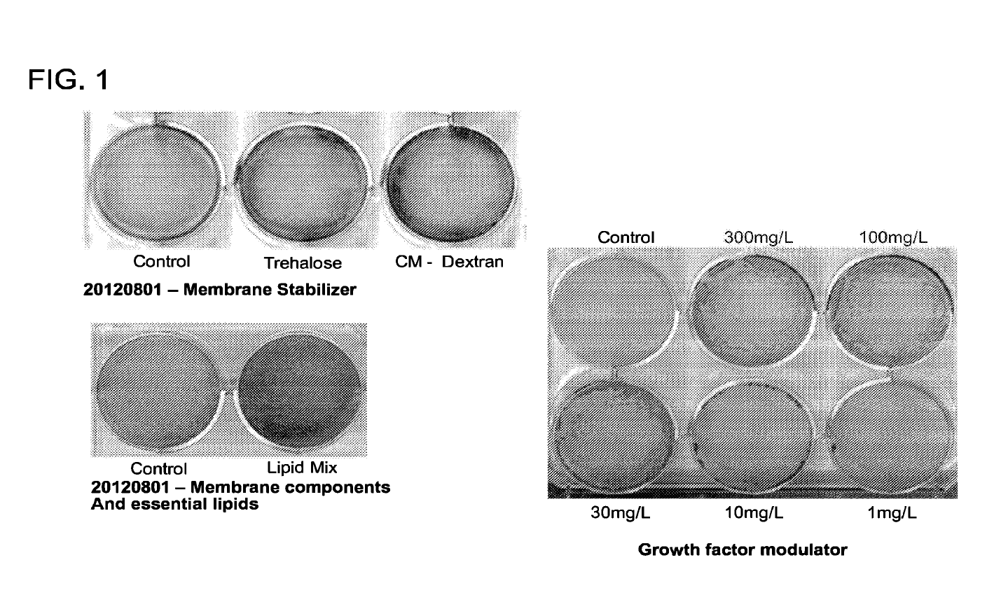

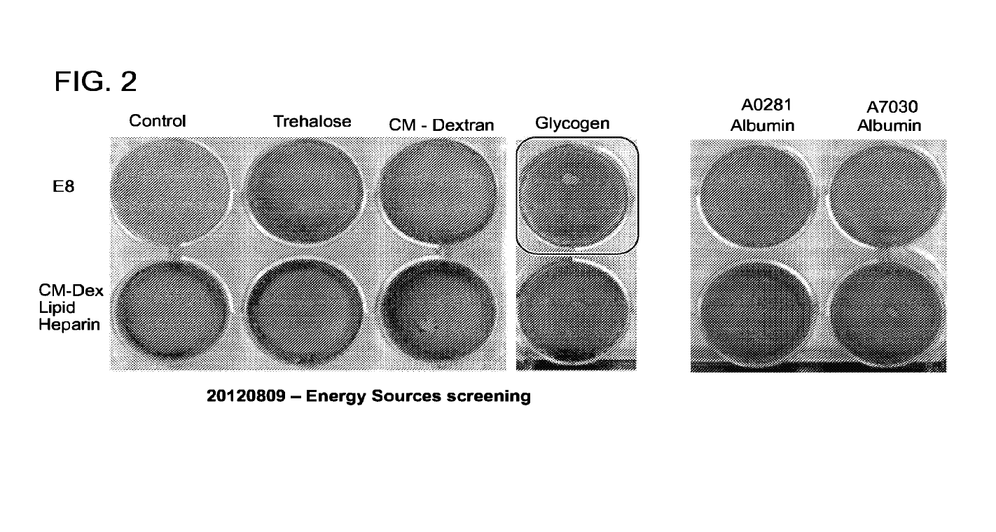

First, initial screening assays were carried out with different additives. In this assay, human embryonic stem cells (ESCs) are grown to near confluence in E8 medium. Culture is continued with different additives as indicated in FIGS. 1 and 2. The additives in the set of experiments described herein are selected from trehalose, CM-Dextran, lipid mix, heparin, glycogen and albumin. Cells that were untreated served as the controls. The protocol is set forth below:

1. Human ESCs were expanded with EDTA dissociation at 1:6 ratio.

2. E8 medium was changed every day.

3. After 2-3 days, when cell density reached around 70-80% confluency.

4. Change medium, and different treatments were applied to the cells in addition to fresh E8 every day.

5. Cells in E8 started to die 1-2 days after they reached 100% confluency.

6. Plates were stained with Alkaline Phosphatase staining (APS) kit (Vector Lab) to determine pluripotency maintenance. After a few days, the plates are stained with Alkaline Phosphatase Staining (APS). The cell viability reagent PrestoBlue can also be used to measure live cells. In this protocol

1. Human ES cells were cultured at 50% confluence in E8 media when polymers were added into the media.

2. After another two days of culturing, the cell viability reagent PrestoBlue (Life Technologies) was added into the media and incubated for one hour.

3. Cell viability was detected by fluorescence using 560 nm excitation and 590 emission. Results from these initial screening assays are shown in FIGS. 1 and 2.

The results of these experiments showed that new factors, in particular, trehalose, glycogen, CM-Dextran and heparin, enhanced pluripotent stem cell survival and differentiation efficiency.

Trehalose (0.2-1%), shown below, is a natural alpha-linked disaccharide formed by an α,α-l,l-glucoside bond between two a-glucose units.

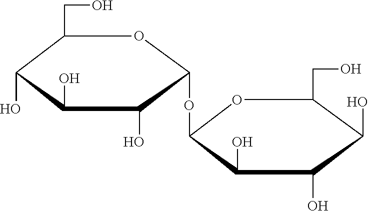

Glycogen (0.1-1%; also called dextrin in older texts, which is not to be confused with the polysaccharide called dextran that is made by bacteria), shown below, is found principally in muscle and liver cells, where it serves as a readily accessible depot for the storage of glucose.

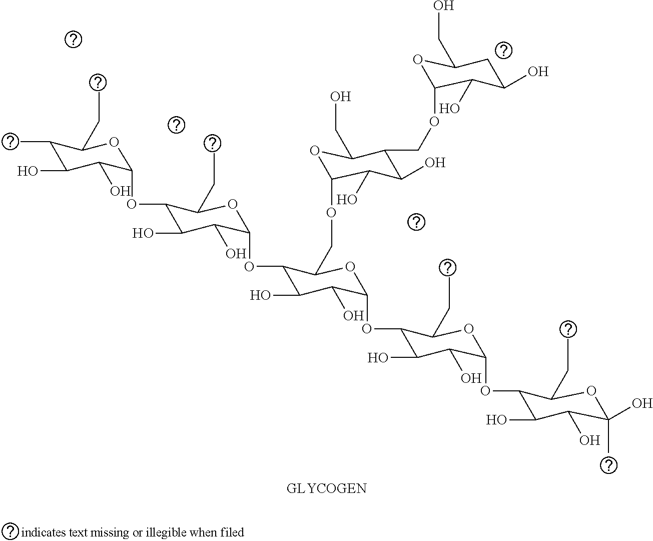 [SMILES: CCC1OC(OC2C(CC)OC(OC3C(COC4OC(CO)C(COC5OC(CO)CC(O)C5O)C(O)C4O)OC(OC4C(CC)OC(OC5C(CC)OC(C)(O)C(O)C5O)C(O)C4O)C(O)C3O)C(O)C2O)C(O)C(O)C1C]

CM-dextran (0.2-1%), shown below, consists of a dextran backbone substituted with carboxymethyl substituents imparting a polyanionic character to the product. The carboxymethyl content corresponds to about 1 CM group for every 5 glucose units.

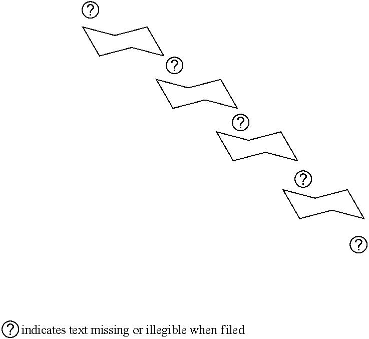

Heparin (20 mg/L), shown below, also known as unfractionated heparin, a highly sulfated glycosaminoglycan, is widely used as an injectable anticoagulant, and has the highest negative charge density of any known biological molecule.

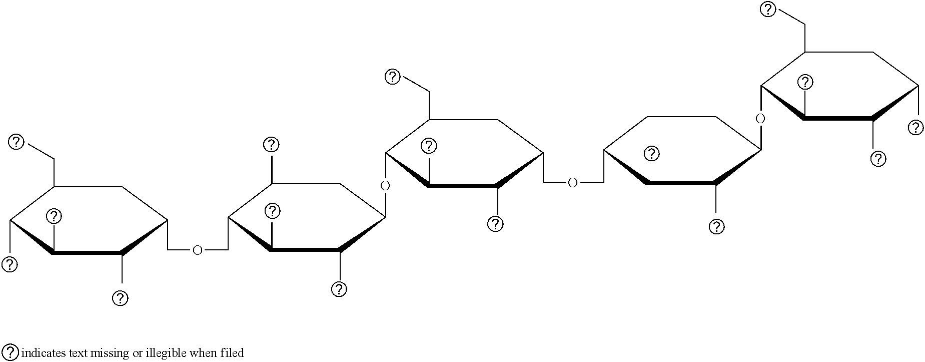

Heparan sulfate is a linear polysaccharide found in all animal tissues. It occurs as a proteoglycan (HSPG) in which two or three HS chains are attached in close proximity to cell surface or extracellular matrix proteins.

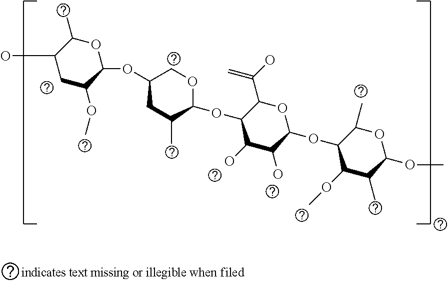

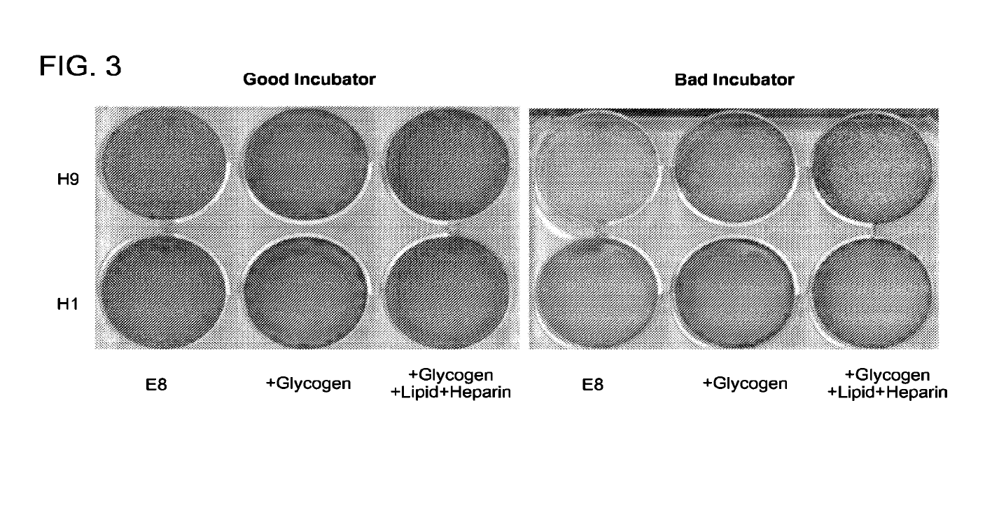

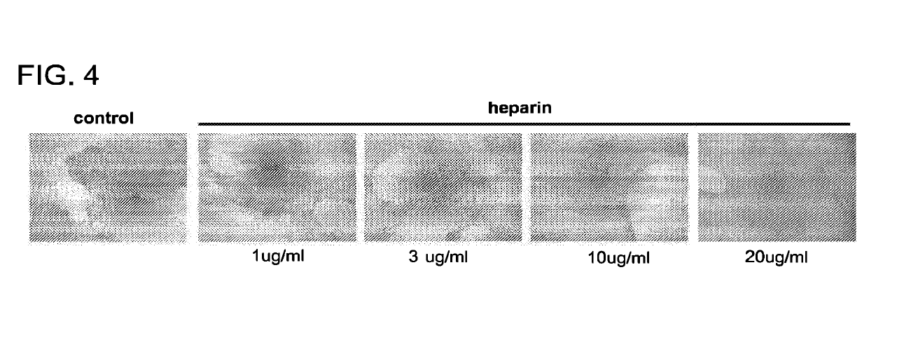

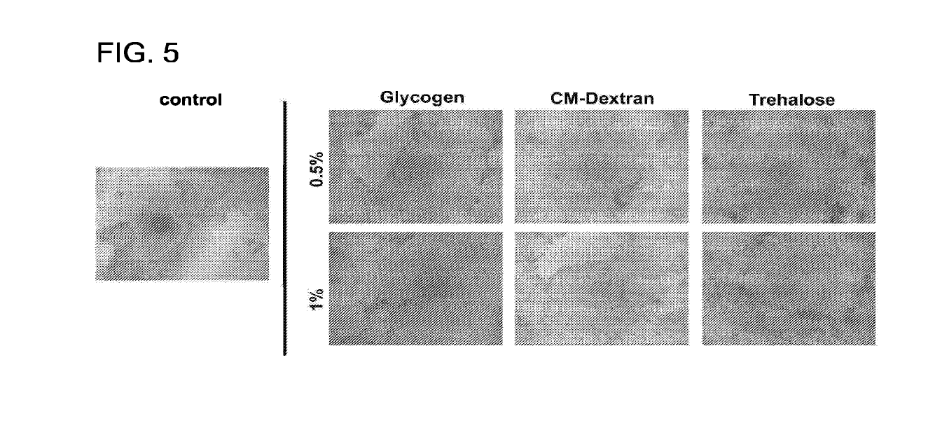

These new factors (e.g. one or more of trehalose, CM-Dextran, lipid mix, heparin, glycogen and) were also beneficial for cell maintenance in suboptimal conditions. As shown in FIG. 3, most cells do grow, albeit slower, and the cells also differentiated even in suboptimal conditions “bad incubator.” The dosages of these factors were also tested for hPSCs survival as shown in FIGS. 4 and 5.

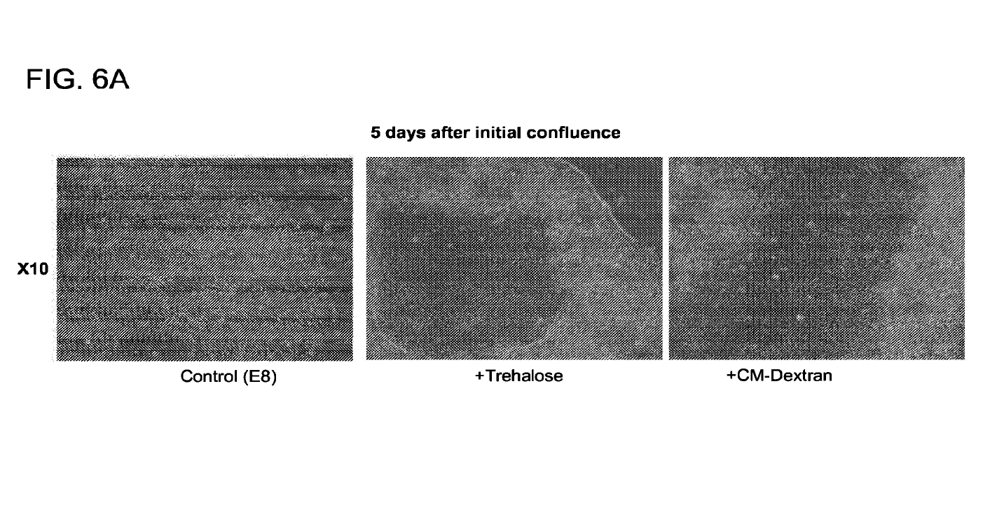

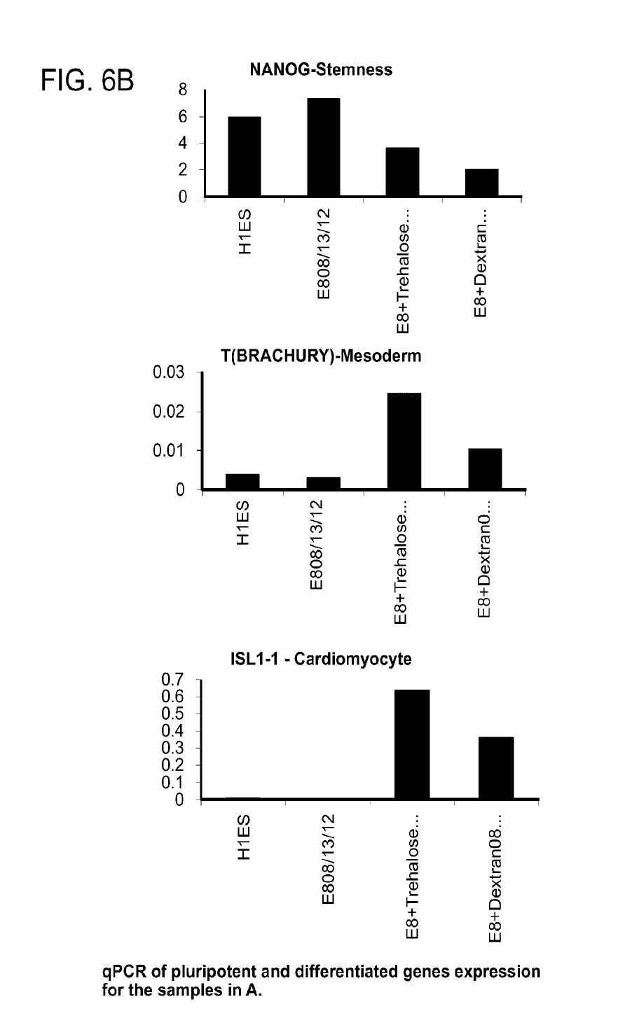

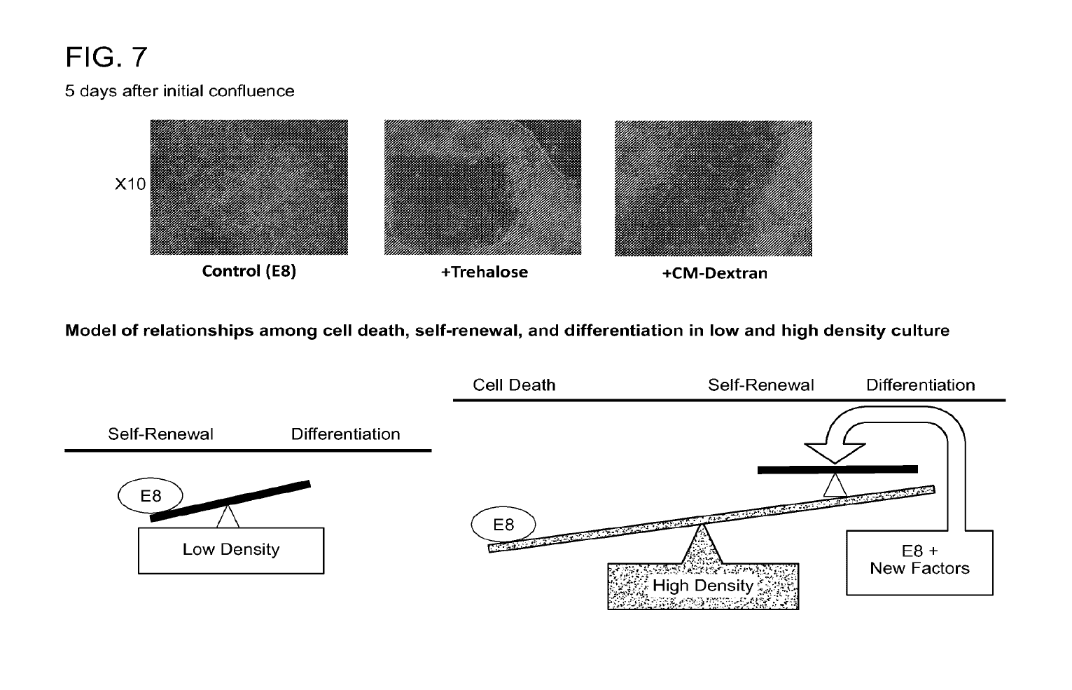

Next, experiments were carried out with cells grown in E8 medium supplemented with one or more polysaccharides (e.g. trehalose, CM-Dextran or glycogen). FIG. 6 shows cell proliferation and spontaneous differentiation at high density with trehalose and CM-dextran. ES cells were cultured in E8 medium supplemented with trehalose or CM-dextran. FIG. 6 shows the cells 5 days after initial confluence. As shown in the control panel, on the left, most cells died and leftover cells grew up again and did not differentiate. However, in the trehalose and CM-dextran supplemented medium, shown in the center and right panels respectively, there was little cell death, and the cells differentiated. The graphs at the bottom of FIG. 6 show that in cells cultured in E8 medium supplemented with trehalose or CM-dextran, markers of differentiation were expressed. All the data suggest that E8 maintains self-renewal and pluripotency in low-density culture. However, in high density, E8 needed supplementation of other factors which help inhibit cell death as well as balance self-renewal and differentiation (FIG. 7).

Most current differentiation protocols use undefined conditions and require high density culture. Cells cultured in E8 medium usually cannot differentiate. Therefore, the problems to be addressed in developing an E8 based differentiation platform were to decrease cell death at high density during differentiation and the difference in growth factor treatment and timing. Nine initial differentiation tests were performed—four with cardiomyoctes, one neural, one hepatocyte, one endothelial, one smooth muscle and one blood lineage.

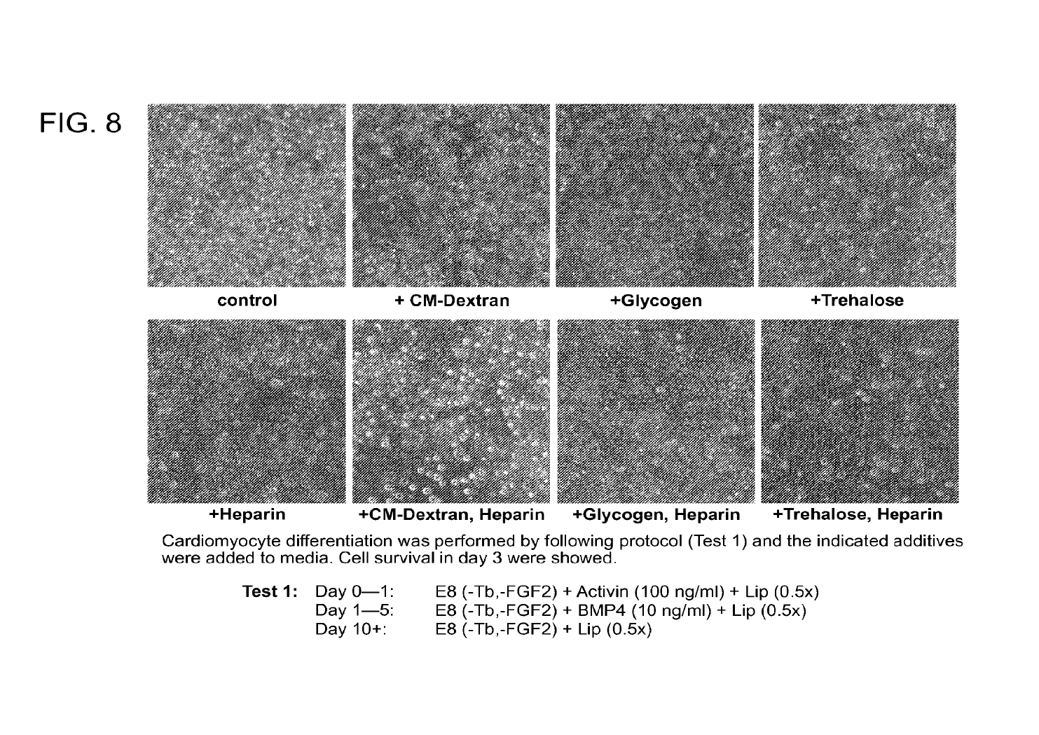

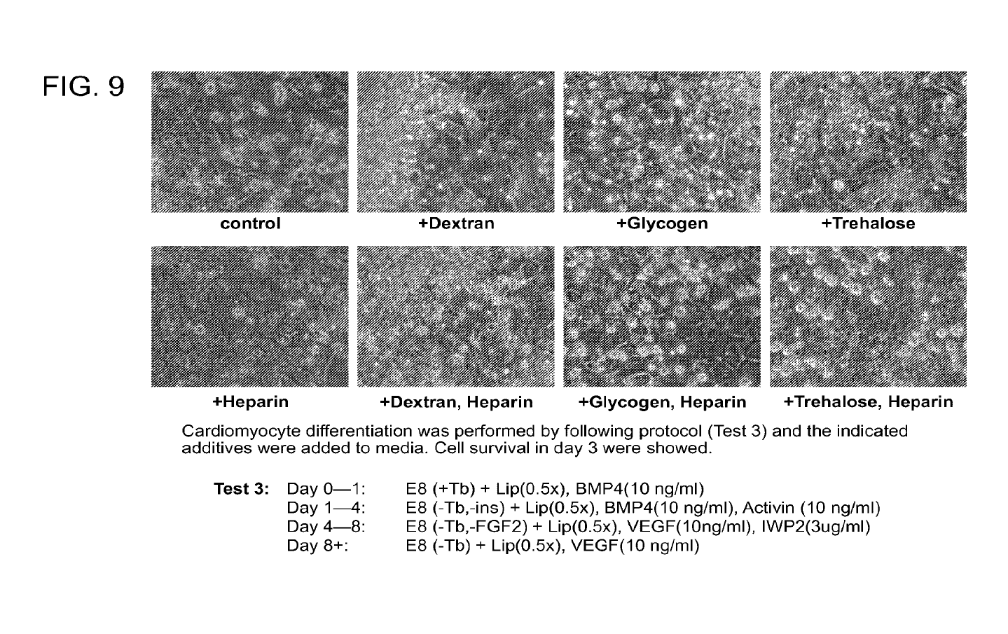

Experiments were carried out to determine if these newly identified factors suppressed cell death during differentiation. FIGS. 8 and 9 show that E8 medium supplemented with the new factors (dextran, glycogen, trehalose, heparin, dextran and heparin, glycogen and heparin, trehalose and heparin) suppressed cell death during differentiation. FIG. 8 shows cardiomyocyte differentiation that was performed by test condition 1 (Test 1), as indicated in the FIG. 8 legend. The indicated additives (CM-Dextran, Glycogen, Trehalose, Heparin, or various combinations were added to the media. Cell survival at day 3 is shown. FIG. 9 shows cardiomyocyte differentiation that was performed by test condition 3 (Test 3), as indicated in the FIG. 9 legend. The indicated additives (CM-Dextran, Glycogen, Trehalose, Heparin, or various combinations were added to the media. Cell survival at day 3 is shown. Cell density is increased in cells cultured in E8 medium supplemented with the new factors (glycogen, trehalose, heparin).

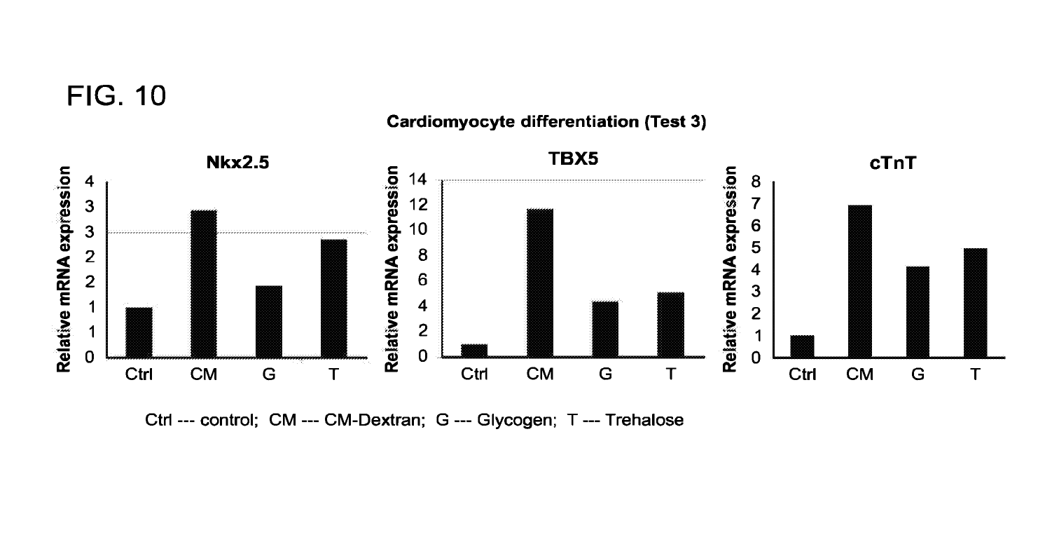

Gene expression during differentiation was examined, and it was shown that the new factors affected specific gene expression during differentiation. FIG. 10 shows the results of qPCR that was performed to assess relative mRNA expression of cardiac markers for cells following the Test 3 protocol, as described infra. FIG. 10 shows that the new factors (CM-Dextran (DM), glycogen (G), trehalose (T), promoted cardiomyocyte differentiation, as shown by mRNA expression of NKX2-5, TBX5 and TNNT2, which are markers of cardiomyocyte differentiation.

### Example 3

### Heparin Promotes Cardiac Differentiation in Chemically Defined Conditions

Cardiomyocytes are thought to be terminally differentiated. Although a small percentage of the cells may have proliferative capacity, it is not sufficient to replace injured or dead cardiomyocytes. Death of cardiomyocytes occurs, for example, when a coronary vessel is occluded by a thrombus and the surrounding cardiomyocytes cannot be supplied with necessary energy sources from other coronary vessels. Loss of functional cardiomyocytes may lead to chronic heart failure. A potential route for restoring “normal” heart function is replacement of injured or dead cardiomyocytes by new functional cardiomyocytes. It would be advantageous to transplant healthy cardiac cells from the patient's iPSCs.

At the same time, heart is often the main target of toxicity of drugs. Therefore, high quality cardiac cells are essential for excluding high-risk drugs. Patient specific cardiac cells could also be used to test drug dose for personalized therapies. It is important to generate large quantities of patient specific cardiac cells that could be used in therapy, diagnosis and screening. Human pluripotent stem cells (hPSCs) offer the potential to generate large numbers of functional cardiomyocytes from clonal and patient-specific cell sources.

Described herein is a new approach using heparin and heparan sulfate that could significantly improve the production of cardiac differentiation from human ESC and iPSCs. These experiments demonstrate that heparin can contribute to cardiac differentiation and that a new cardiac differentiation culture improves the efficiency of cardiomyocyte differentiation. It is expected that these culture conditions will be applicable to all hES cell lines and hiPSCs in general. Furthermore, the fact that these differentiation conditions are established without fetal calf serum, and thus without the presence of animal pathogens, increases the chance that these hES-derived cardiomyocytes are suitable for cardiomyocyte transplantation in patients with heart disease.

Heparin and Heparan Sulfate regulate growth factor activities, for example FGF, WNT, BMP. Further, defects in heparan sulfate synthesis lead to deficiency in cardiac differentiation. Other protocols described in the art use undefined factors and have inconsistent production depending on batch quality of source materials. For example, B27 medium is a complex medium that has shown to have quality issues and inconsistencies, for example inconsistent albumin concentrations. The present invention provides a simple and well-defined system to produce large quantities of human cardiac cells from iPSC/ESCs.

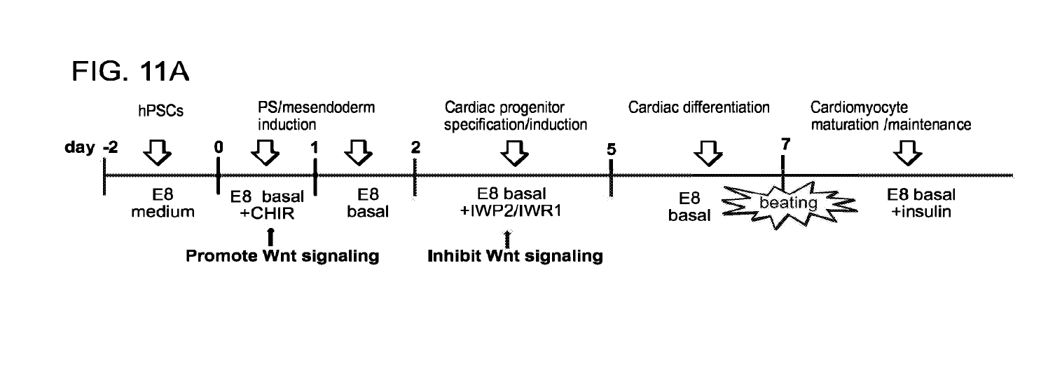

FIG. 11 shows E8 based conditions for cardiac production. As shown in FIG. 11, hPSCs were cultured in E8 medium. CHIR (CHIR99021, a glycogen synthase kinase (GSK)-3a inhibitor) was added to the E8 basal medium to promote WNT signaling and primitive streak (PS)/mesoderm induction (days 0-1). Next, IWP2/IWR1 was added to E8 basal medium to inhibit WNT signaling and promote cardiac progenitor specification/induction (days 2-5). The medium was changed to E8 basal medium (days 5-7) to promote cardiac differentiation. At day 7, beating of the cardiomyocytes could be detected. Cardiac maturation/maintenance was carried out in E8 medium supplemented with insulin. However, this E8 based cardiac differentiation protocol yielded a lower than 50% derivation, and was inconsistent.

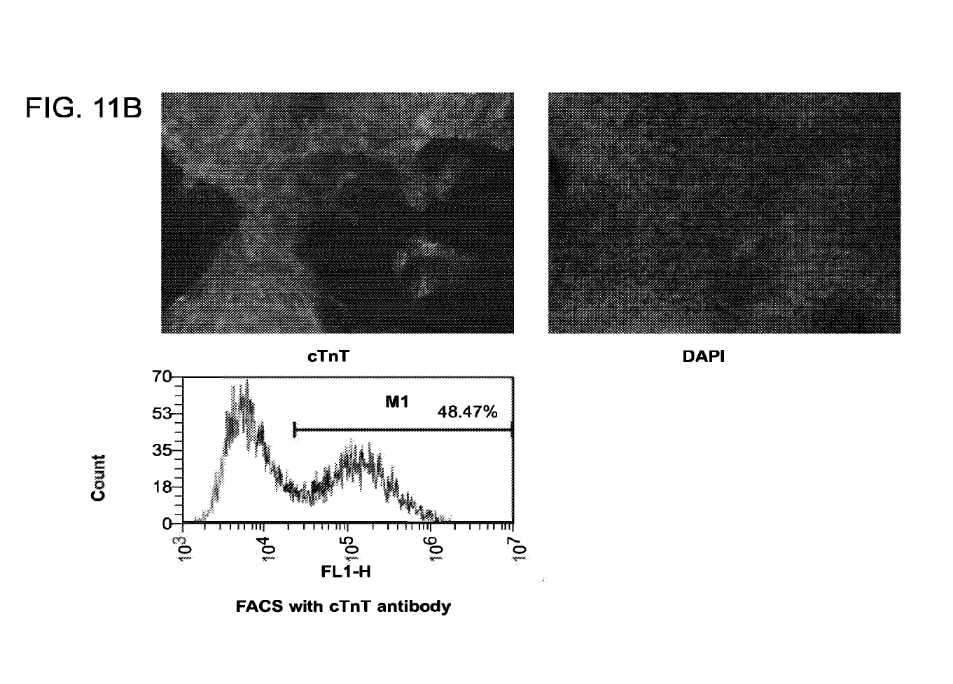

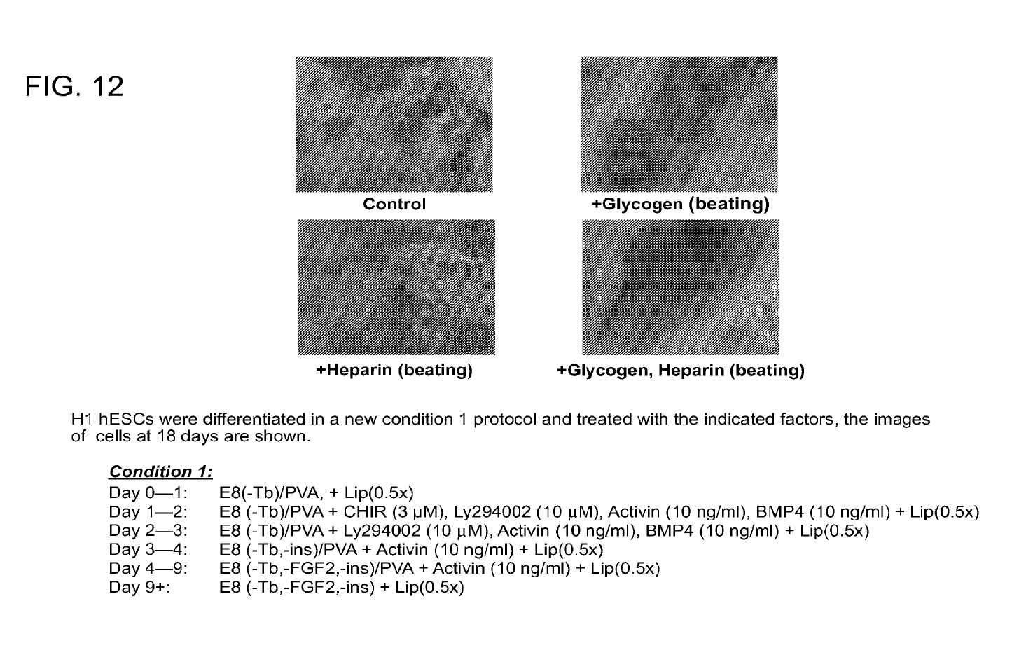

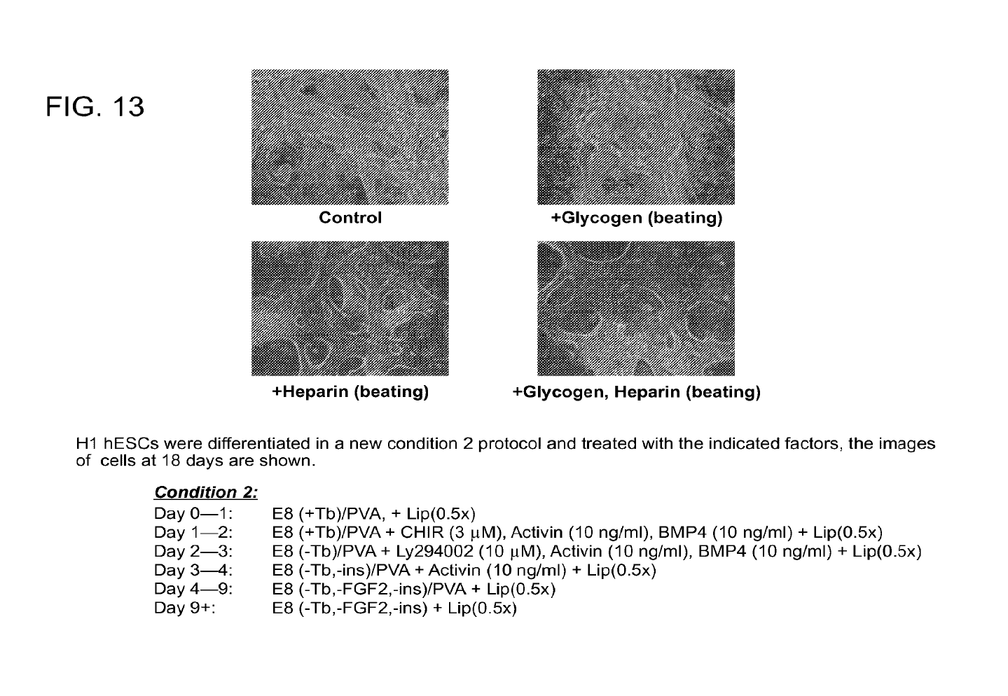

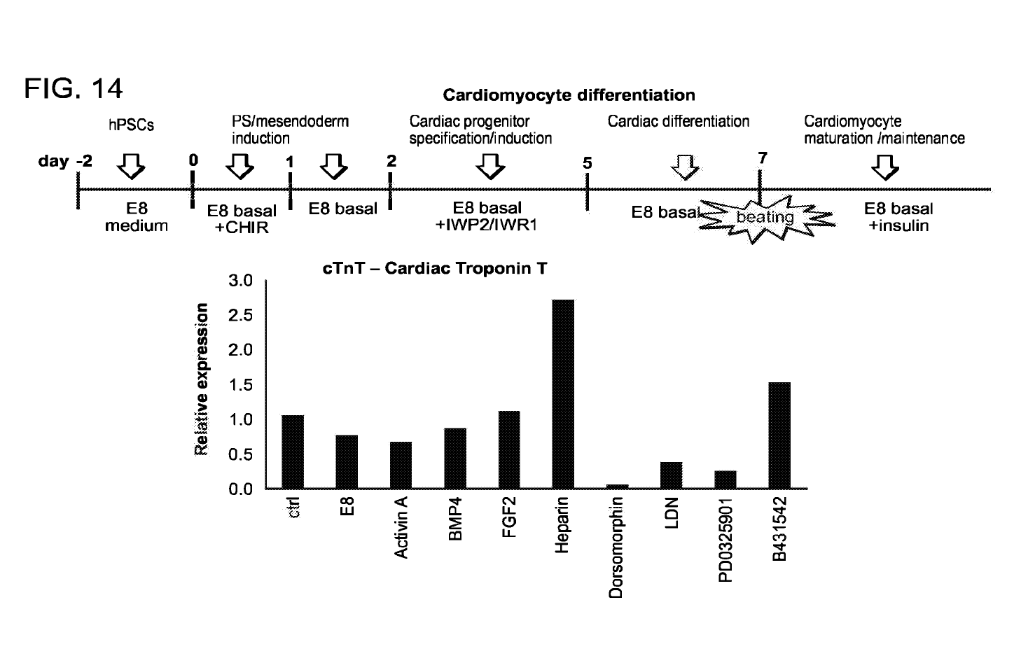

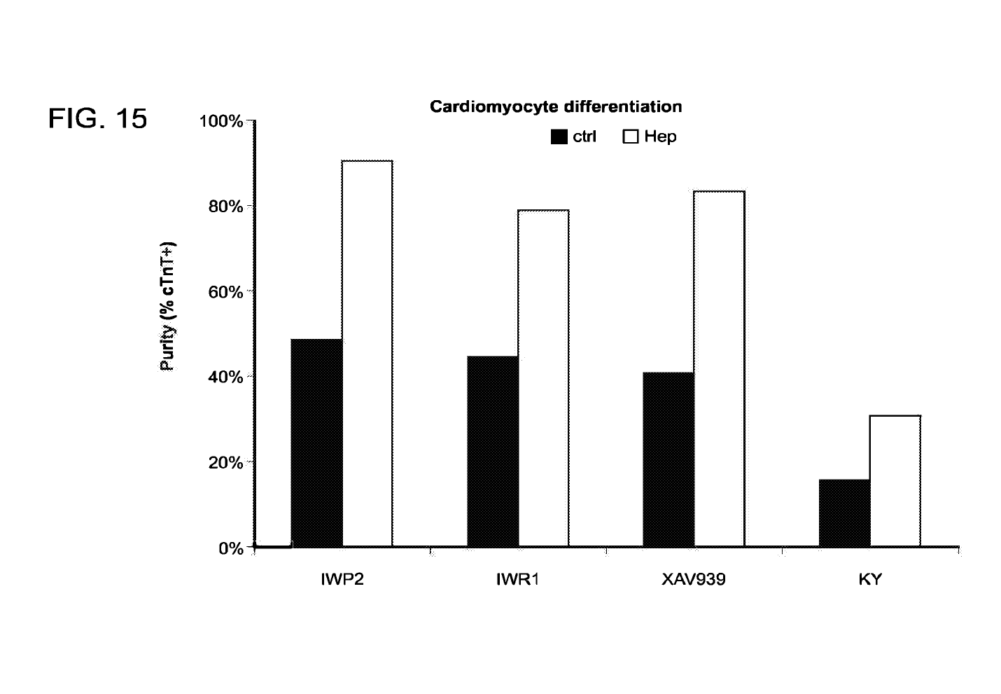

FIGS. 12 and 13 show that the new factors (Glycogen and Heparin) considerably improved cardiomyocyte differentiation in two different conditions, with/without LY294002 inhibitor. The beating cardiomyocyte foci were imaged by microscopy and video camera. Next, screening for factors promoting cardiac differentiation was performed. As shown in FIG. 14, growth factors or inhibitors were added at day 0-1 with CHIR treatment during differentiation. mRNA from 10 days differentiated cells was collected for qPCR of cardiac Troponin T (cTnT). The results are shown in the graph in FIG. 14. When Heparin was added, relative expression of cTnT was considerably increased (similar results were observed for heparin sulfate). Heparin was also shown to be beneficial in combination with different WNT modulators. FIG. 15 shows fluorescence-activated cell sorting (FACS) with cTnT of cells that were differentiated for 10 days with different WNT inhibitors (IWP2, IWR1, XAV939, KY). As shown in FIG. 15, heparin treated cells promoted cardiac differentiation, as seen about 90% cTnT positive cells determined by FACS. This treatment can also be applied to cells in suspension culture and heparin and heparin sulfate would be effective at promoting differentiation even in suspension cultures.

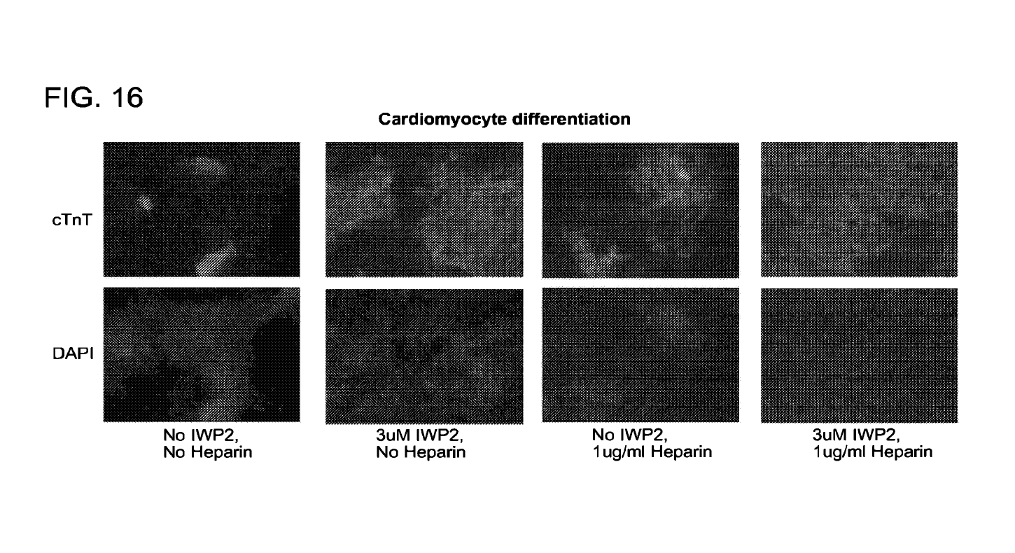

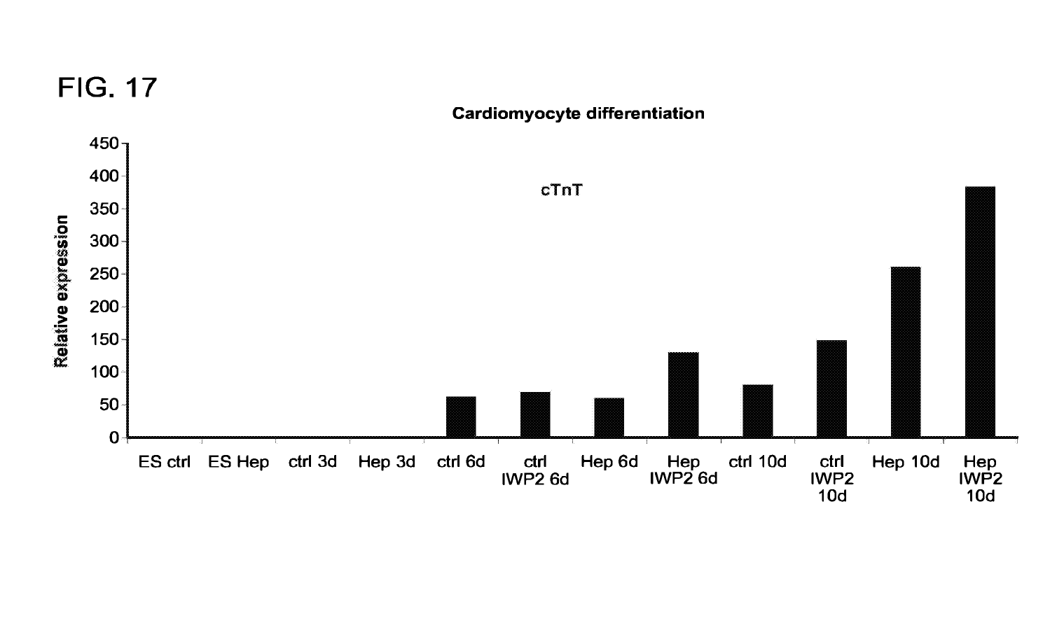

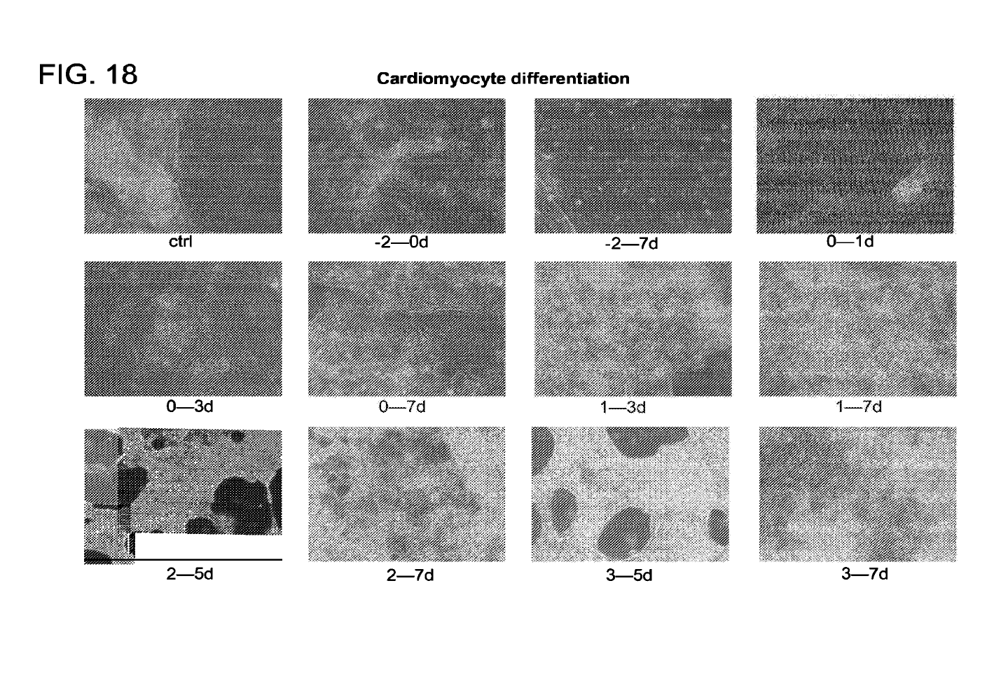

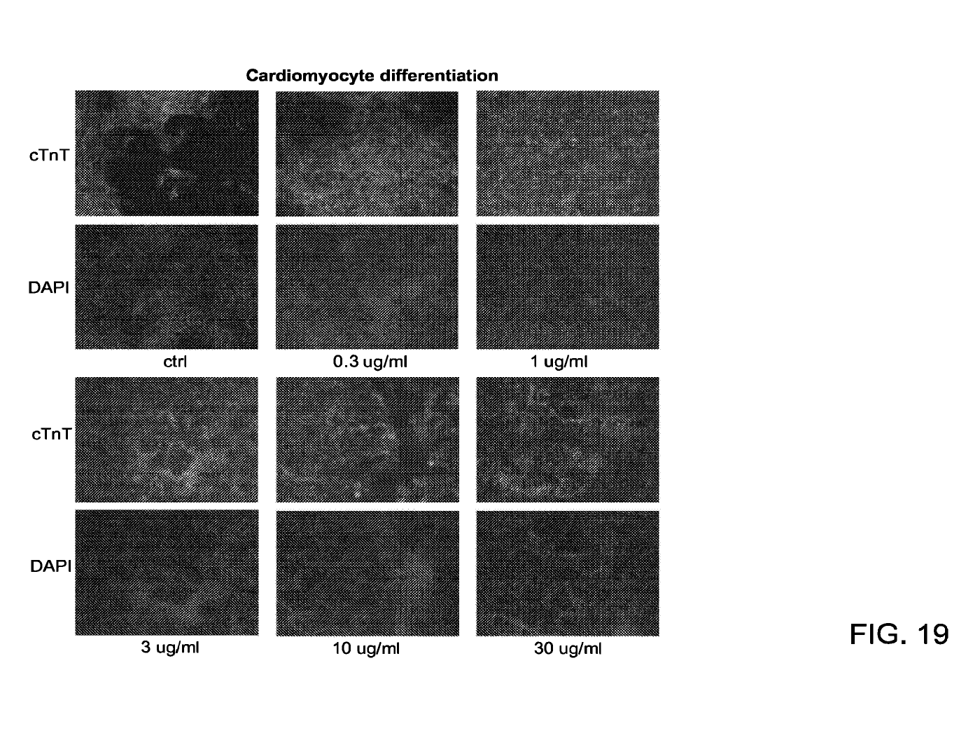

As shown in FIG. 16, it was found that heparin promotes cardiac differentiation in the absence of WNT inhibition. The panel on the left shows control, untreated cells (no IWP2, no heparin). The other panels show cells treated with 3 μM PWP2 and no heparin, no PWP2 and 1 μg/ml heparin, and 3 μM of Wnt inhibitor IWP-2 and 1 μg/ml heparin. As shown in FIG. 16 with cTnT staining, cardiac differentiation was seen without IPW2 and in the presence of 1 μg/ml heparin. FIG. 17 shows the results of microarray experiments demonstrating heparin induced cardiac differentiation (shown by cTnT expression) independent of WNT inhibitors. It was determined through these experiments that the timing of heparin administration affected the extent of cardiomyocyte differentiation. As shown in FIG. 18, cells were treated with 1 μg/ml at different time periods during cardiomyocyte differentiation (−2-0 d; −2-7 d; 0-1 d; 0-3 d; 0-7 d; 1-3 d; 1-7 d; 2-5 d; 2-7 d; 3-5 d; 3-7 d). The results indicated that heparin treatment on days 1-7 promoted cardiomyocyte differentiation. Different doses of heparin (from 0.3 to 10 μg/ml) were tested for their effects on cardiomyocyte differentiation. As shown in FIG. 19 with cTnT staining, 1 μg/ml was identified as a particularly advantageous dosage of heparin for enhancement of cardiomyocyte differentiation.

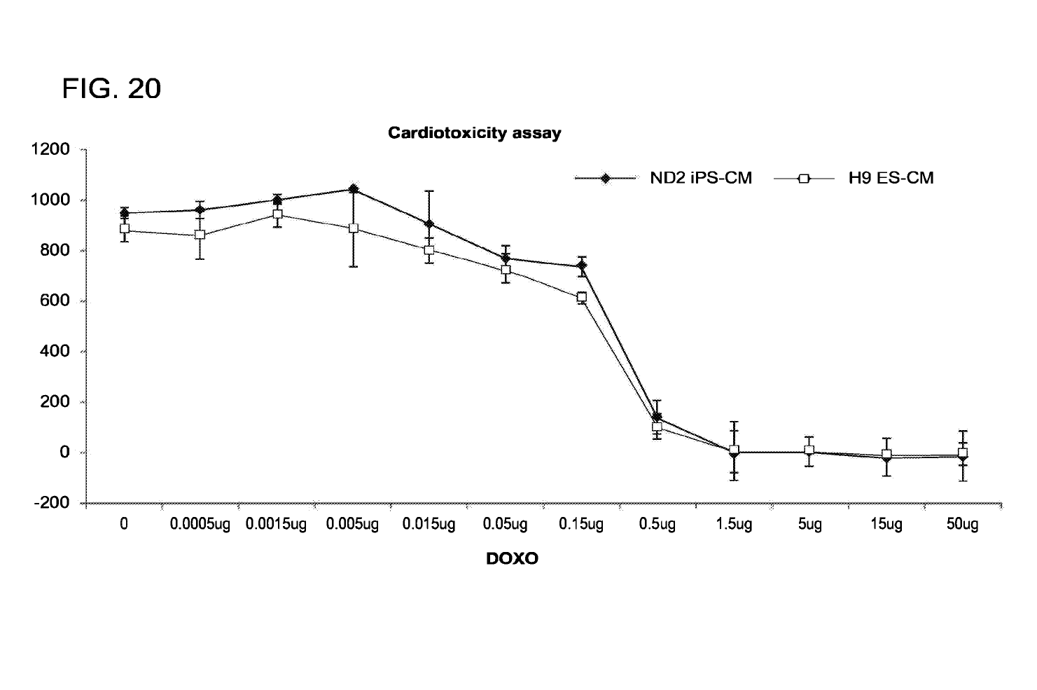

One application of this new approach using heparin and heparan sulfate to improve the production of cardiac differentiation from human ESC and iPSCs is in a cardiac toxicity assay, where a drug is administered to cardiac cells. FIG. 20 shows a doxycycline (Dox) killing curve. Cardiomyocytes were treated with doxocycline at indicated concentrations for 2 days.

Another application is in transplantation. As shown in FIG. 21, human iPSC induced GFP-beating cardiomyocytes were generated. Two weeks after injection into the mouse heart, the GFP cells could be observed as beating. Even 5 weeks after injection, GFP was still visible in the heart (data not shown).

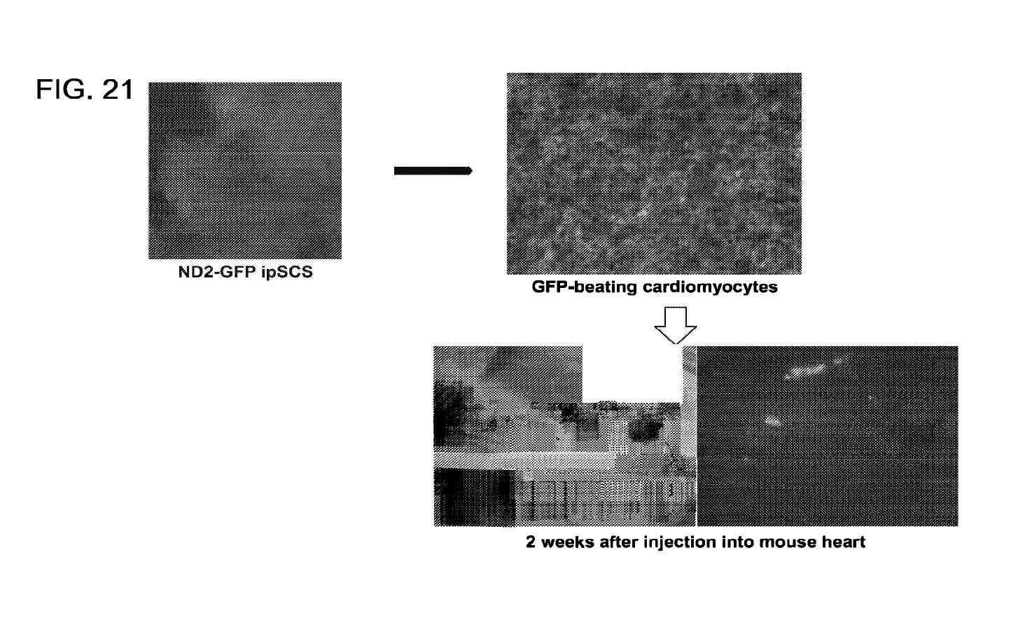

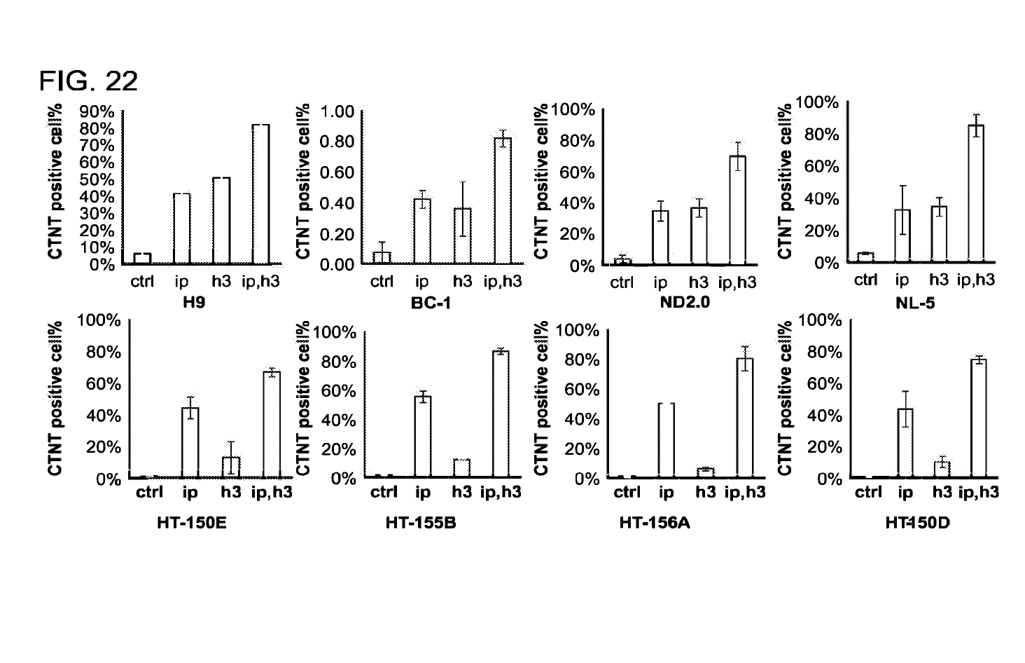

The general function of heparin on the following human ESC/iPSC lines was also demonstrated: H9, BC-1, ND2.0, NL-5, HT-150E, HT-155B, HT-156A and HT-150D (FIG. 22). In such experiments, the effect of heparin on cardiac differentiation was tested, using Troponin T (CTNT) as a marker. A positive effect of heparin was observed.

As described herein, the present invention allows for efficient production of cardiac cells for transplantation and drug discovery. Advantageously, the invention provides a fully defined system for cardiac differentiation, and it is suitable for clinical applications. The high efficiency and consistency in this invention are attractive and necessary for industrial-format mass production. This new discovery simplifies the culture system for cardiac differentiation, and could serve as a new platform for further technology development. The invention can also be used to produce clinical grade cardiac cells.

### Example 4

As noted above, cardiomyocytes derived from hPSCs have been in great demand because of their applications in drug screening and evaluation of cardiac cytotoxicity, as well as their potential for cellular replacement therapy. To generate large numbers of cardiomyocytes for such screening and cell therapy efforts, multiple serum containing, or serum-free, differentiation conditions have been reported in the past decade (Burridge et al., Cell stem cell 2012, 10(1):16-28; Yang et al. Nature 2008, 453(7194):524-28; Lian et al., PNAS 2012, 109(27):E1848-57; Dubois et al., Nature biotechnology 2011, 29(11):1011-18; Ni T T, Chemistry & biology 2011, 18(12):1658-68; Burridge et al., Nature methods 2014, 11(8):855-60; Laflamme et al., Nature biotechnology 2007, 25(9):1015-24). Despite different growth factor and inhibitor combinations used in various serum-free procedures, most conditions contain albumin or albumin-containing supplements, such as B27. These ingredients either contain animal products (Bovine Serum Albumin), or serum substitutes that may produce variations in differentiation efficiency due to batch-to-batch inconsistencies of the source materials. Albumin-free conditions were recently reported with careful modulations of WNT pathway activities during differentiation (Lian et al., Nature methods 2015, 12(7):595-6), but additional cell culture media compositions that improve the efficiency and consistency of cardiomyocyte differentiation in albumin-free conditions are still desired. The inventors have previously developed albumin-free E8 medium for hPSC derivation and maintenance (Chen et al. Nature methods 2011, 8(5):424-29). These E8-based media provide a suitable platform from which to begin screening for novel albumin-free differentiation conditions for hPSC cardiomyocyte differentiation.

Albumin, a major serum component, has long been used in somatic and stem cell culture and plays important roles on cells in culture. Albumin is not only a carrier for lipids and other bioactive factors, such as vitamins and fatty acids, but is also involved in biophysical modulation as a protective reagent and colloid osmolality regulator. The complicated roles of albumin in cell culture often prevent the discovery of other important regulatory factors in stem cell culture. DMEM/F-12 based E8 medium is an albumin-free stem cell medium that serves as a good starting point to develop albumin-free differentiation conditions for cardiomyocytes, but there have been no novel protocols based on the E8 platform. In this study, the inventors have identified novel cell culture compositions and conditions and efficient methods for hPSC cardiac differentiation using an albumin-free E8 medium platform.

In this research, heparin was identified as an important factor promoting cardiomyocyte differentiation in the absence of albumin. Signaling pathway analysis indicated that heparin modulated WNT to promote cardiomyocyte differentiation. Based on these results, the inventors optimized a robust cardiomyocyte differentiation protocol that has been reproducibly applied on more than 10 hESC and hiPSC lines. This differentiation methodology is the most cost-effective protocol to date that produces comparable efficiency and consistency of cardiac differentiation as previously-reported protocols. This protocol can provide large-scale, high quality hPSC-derived cardiomyocytes at low cost for drug screening and cellular therapies.

### Experimental Procedures

hPSC cell maintenance: Human ESCs (H1 and H9) and iPSCs were maintained in E8 media (Essential 8 Medium, Life Technologies) on MATRIGEL™-coated (10 μg/cm²) plates with daily medium changes. Cells were passaged with EDTA. Briefly, 60-70% confluent hPSCs were washed with 0.5 mM EDTA/PBS once and incubated in 0.5 mM EDTA/PBS for 3-5 minutes at room temperature. EDTA/PBS was removed from the plate and the cells were gently washed off via E8 medium containing Rock inhibitor and passaged onto MATRIGEL™-coated plates at 1:6-1:8 dilutions. Rock inhibitor was removed on the next day after passage.

Cardiomyocyte differentiation in monolayer: When hPSCs had grown to 80-90% confluent, 2-3 days after plating, the medium was changed from E8 to differentiation basal medium, which contains E8 basal medium (DMEM F-12, L-ascorbic acid, Selenium, Transferrin, and NaHCO₃), 1× Chemically Defined Lipid Concentrate (100×, Life Technologies, 11905-031; a commercially-available lipid mixture of Arachidonic Acid, Cholesterol, DL-α-Tocopherol Acetate, Ethyl Alcohol 100%, Linoleic Acid, Linolenic Acid, Myristic Acid, Oleic Acid, Palmitic Acid, Palmitoleic Acid, Pluronic F-68, Stearic Acid, Tween 80™) and 1× Pen Strep (Life Technologies, 15140-122). That day was defined as day 0. CHIR99021 (5 uM, Tocris, 4423) was added into differentiation basal medium at day 0 for 24 hrs. IWP2 (3 uM, Tocris, 3533) was added from day 2 to day 5. Heparin was added into the medium at indicated dosages and timeline. Insulin (20 ug/ml, Sigma, 19278) was added into differentiation basal medium to maintain cardiomyocytes from day 7 onward. The medium was changed daily until day 7, and then changed every 2-3 days from day 7 onward. The growth factors, inhibitors, and enzymes used in culture were: Activin A (10 ng/ml, R&D, 338-AC/CF), BMP4 (10 ng/ml, R&D, 314-BP/CF), FGF2 (100 ng/ml, Peprotech, 100-18B), TGFβ1 (1.74 ng/ml, R&D, 240-B/CF), Dorsomorphin (3 μM, Stemgent, 04-0024), LDN-193189 (0.1 μM, Stemgent, 04-0074), PD0325901 (1 μM, Stemgent, 04-0008), SB431542 (3 μM, Selleckchem, s1067), Heparin (Sigma, H3149), Heparinase I (New England Biolabs, P0735S).

Real-time PCR: RNA was purified using TRI Reagent™ Solution according to Ambion's protocol. Residual DNA was removed using the TURBO DNA-Free™ kit. Reverse transcription was carried out with Maxima H Minus Reverse Transcriptase (Thermo Scientific) primed with Poly N15-mer (Eurofins) with the recommended protocol. Prior to PCR, RNA template was removed with addition of Ambion Ribonuclease H from E. coli. Real-time PCR was performed on the BIO-RAD CFX96 using SsoAdvanced™ Universal SYBR® Green Supermix. PCR primers were listed in the supplementary table.

Immunofluorecence staining: Cells cultured in monolayer were fixed in 2% PFA for 10 minutes at room temperature, washed with PBS and permeabilized with 0.2% Triton X-100/PBS for 3 minutes. After successive washes in PBS, the cells were blocked in 10 mg/ml BSA for 30 minutes and incubated with primary antibody for 1 hour at room temperature. Cells were then washed 3 times with PBS for 5 minutes and incubated with secondary antibodies (Alexa 488 or 594-conjugated) for 1 hour. DAPI was applied for nuclei staining. The following primary antibodies were used for immunofluorescence: alpha actinin (mouse, 1:1000, A7811, Sigma), CTNI (cardiac troponin I, rabbit, 1:200, sc-15368, Santa Cruz), CTNT (cardiac troponin T, mouse, CT3, 1:1000, DSHB), Desmin (mouse, 1:100, M0760, DaKo) NKX2.5 (rabbit, ab35842; 1:200, Abcam).

Flow cytometry assay: Cells differentiated for 10 days in monolayer were dissociated with TrypLE (Invitrogen) for 5 minutes. Single cells were washed off and suspended by DMEM/10% FBS followed by centrifugation. The pellets were washed by PBS once and fixed by 2% PFA for 5 minutes. After washing with PBS and centrifuging, permeabilization was performed by 0.3% Triton X-100 for 3 minutes. The cells were centrifuged and re-suspended with 1% BSA to block unspecific binding of antibody, followed by incubation with primary antibodies (diluted in 1% BSA) for 1 hour. After washed, the cells were incubated with second antibodies conjugated with FITC or PE (Invitrogen, 1:1000 diluted in PBS) for 30 minutes. The cells were washed once and suspended in PBS for the flow cytometry assay.

Electrophysiological Assay: Cardiomyocytes were dissociated and plated on glass coverslips in culture medium before use. For action potential measurements, cells were placed in a small-volume chamber and bathed in Tyrode solution containing (mM) 140 NaCl, 5.4 KCl, 1.8 CaCl₂, 1 MgCl₂, 10 HEPES 10 glucose, pH 7.4, maintained at 33-35° C. Action potentials were recorded using a patch clamp amplifer (Axopatch 200B, Molecular Devices) in whole-cell current-clamp mode. Pipettes (1-3 MOhm) were filled with internal solution containing 100 K-aspartate, 30 KCl, 10 HEPES 1 MgCl₂, 5 Mg-ATP, 5 Na₂ creatine phosphate, 0.1 EGTA, 0.025 CaCl₂ (free Ca₂+50 nM), pH 7.2.

Microarray Analysis: Global gene expression data was generated per sample using Agilent's One Color Gene Expression Oligo arrays in accordance with the manufacturer's guidelines. Statistical analysis of the data was performed in R (cran.r-project.org/). Raw expression data generated across samples were first pedestaled by 2, Log 2 transformed, then quantile normalized. After, quality of the data was assured via sample-level Tukey box plot, covariance-based PCA scatter plot and correlation-based Heat Map. Gene probes not having at least one expression measurement greater than system noise were deemed “noise-biased” and discarded. System noise was defined as the lowest observed expression value at which the LOWESS (locally weighted scatter plot smoothing) fit of the data (CV˜mean) grossly deviates from linearity. For gene probes not discarded, expression measurements were floored to equal system noise if less than system noise and the LOWESS fit itself used in conjunction with the random normal distribution to construct four virtual expression values per actual observed value. These values were then subject to one factor ANOVA (analysis of variance) testing under BH (Benjamini and Hochberg) FDR (false discovery rate) MCC (multiple comparison correction) condition using Time::Protocol as the factor. Gene probes found to have a corrected P-value>=0.05 by this test were discarded while remaining probes were subject to the Tukey HSD (honestly significant difference) post-hoc test. Gene probes with a post-hoc P-value<0.05 by and a difference of means 1.50 were subset as having expression “significantly different” between the Time::Protocol conditions being compared. Annotation of subset gene probes was accomplished using IPA (Ingenuity, Inc.). For gene probes annotated in IPA as cardiogenesis-related, expression at Day 6 and Day 10 was subset and compared via Pearson Correlation with the average expression observed at Day 6 and Day 10 for probes representing genes “TNNT2” and “NKX2-5”. Gene probes found to have a significant (P<0.05) positive correlation were summarized by Heat Map and construed to be the cardiogenesis genes involved at Day 6 and Day 10. IPA was also used to explore and compare gene expression differences ongoing in the Wnt signaling pathway at Day 6 and Day 10. Differences in this pathway were also summarized by Heat Map.

Myocardial Infarction and Cardiomyocyte Injection: Myocardial infarction was performed by ligation of the left anterior descending coronary artery in NSG mice (Jackson Laboratory) under anesthesia of 1-3% isoflurane. hPSCs-derived cardiomyocytes (5×10⁵/20 μl) were injected into the myocardium at the border zone of the infarct area through 29-gauge needle. Seven weeks post surgery, the animals were euthanized and the hearts were removed and perfused with 4% paraformaldehyde for immunostaining.

Statistical Analysis: All data are presented as the mean±SD of 3 or more independent experiments. Significance was determined by Students t-test.

### Results

Heparin Enhances hPSC Cardiomyocyte Differentiation Along with Wnt Modulation in E8 Medium Platform

In order to develop an albumin-free condition for cardiomyocyte differentiation, the inventors first adapted singular modulation of WNT signaling, which was previously reported to show high efficiency when adding the BSA (Bovine Serum Albumin)-containing B27 supplement (Lian et al., PNAS, 2012, supra), to an E8 medium platform. E8 is a DMEM/F-12-based, albumin-free medium containing: L-ascorbic acid, selenium, transferrin, NaHCO₃, insulin, bFGF, and TGFβ (see, for example, Chen, et al., Nature methods 2011, 8(5):424-29).

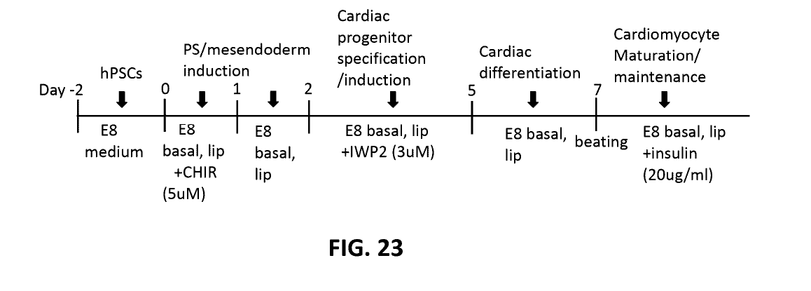

The inventors developed an E8 basal medium (consisting of E8 media without NaHCO₃, insulin, bFGF, or TGFβ; i.e., containing DMEM/F-12, L-ascorbic acid, selenium, and transferrin). This E8 basal medium was used in a differentiation protocol for human H1 ESCs with initial WNT activation (using GSK3β inhibitor, CHIR99021) applied to the cells in culture at days 0-1 (i.e., days following initiation of the stem cell differentiation protocol; day 0 is the day on which the stem cell differentiation protocol begins when the previously plated cells reach at least 75% confluence and the specific cell culture media are applied to the cells to being the cardiomyocyte differentiation protocol, day 1 is the next day in which specific media may be applied to the cells, and day 2 is the following day, etc.). In this protocol, WNT activation at days 0 and 1, is followed by WNT suppression (using WNT secretion inhibitor, IWP2) on days 2-5. Albumin or B27 were specifically excluded from the media used during the entire protocol. A diagram of this protocol is shown in FIG. 23. Beating cardiomyocytes emerged at day 7 after differentiation without the addition of B27 supplement or human recombinant albumin, but rarely exceeded 50% purity at day 10, indicating that additional regulation is necessary for more robust cardiomyocyte differentiation. These data also indicate that this protocol can be used as a screening platform to identify additional differentiation-promoting regulators in the absence of albumin.

Multiple signaling pathways (TGFβ, BMP, FGF, Activin/Nodal) crosstalk with Wnt signaling in regulating mesoderm induction and cardiac differentiation. The inventors therefore sought to test whether these growth factors, or their specific inhibitors, altered WNT-modulated cardiac derivation at different differentiation stages in this E8 basal media-based platform.

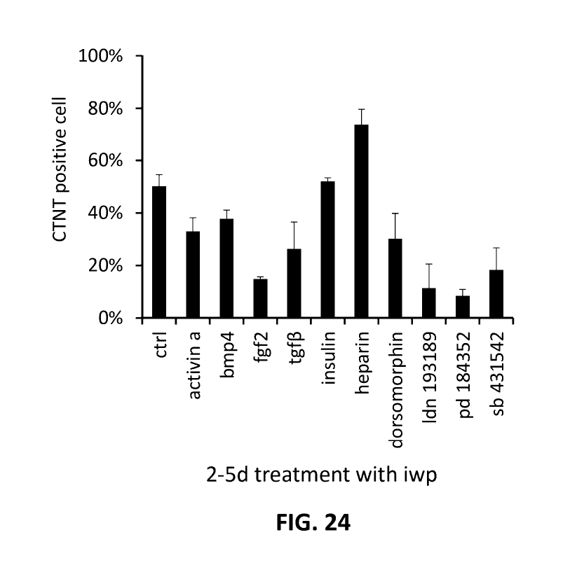

Heparin was also added to the compound screening list as this proteoglycan plays an essential role in interacting and regulating multiple growth factor signaling pathways. As shown in FIG. 24, the following growth factors and inhibitors were tested (at the indicated concentrations): Activin A (10 ng/ml, R&D, 338-AC/CF), BMP4 (10 ng/ml, R&D, 314-BP/CF), FGF2 (100 ng/ml, Peprotech, 100-18B), TGFβ1 (1.74 ng/ml, R&D, 240-B/CF), Dorsomorphin (3 uM, Stemgent, 04-0024), LDN-193189 (0.1 uM, Stemgent, 04-0074), PD0325901 (1 uM, Stemgent, 04-0008), SB431542 (3 uM, Selleckchem, s1067), Heparin (Sigma, H3149), Heparinase I (New England Biolabs, P07355).

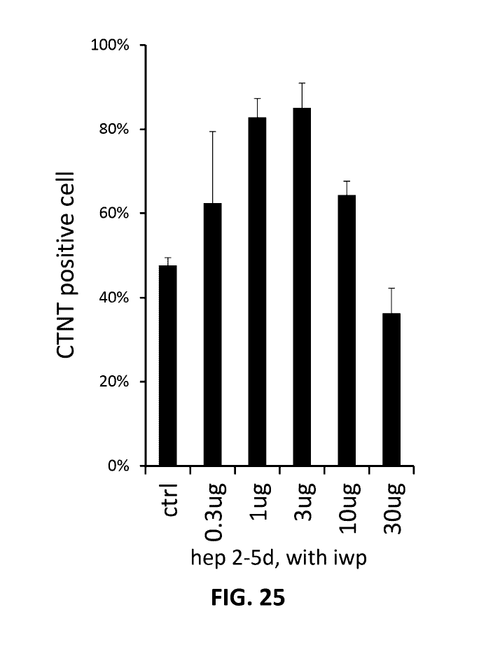

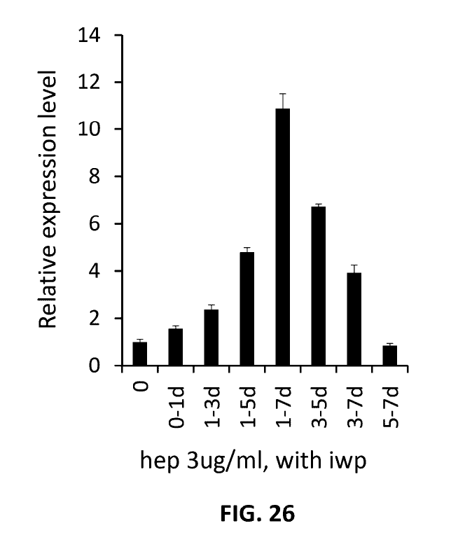

Modulation of conventional signaling pathways by growth factors (TGFβ, BMP, FGF, Activin/Nodal), and their specific inhibitors, did not improve cardiomyocyte differentiation efficiency, which is consistent with the consensus that WNT inhibition is the main player promoting cardiac differentiation after mesoderm induction. Treatment of the cells with heparin from day 2 to day 5 significantly enhanced the efficiency of cardiac differentiation (FIG. 24). This effect of heparin treatment was surprising and unexpected, and probably does not result from the effects of heparin acting through FGF pathways because neither the FGF2-, nor the FGF-pathway inhibitors improved cardiomyocyte differentiation in the screen. This significant impact of heparin on cardiac differentiation had not been previously reported, so the inventors further confirmed this effect by evaluating the impact with time-course and dosage experiments. Heparin treatment from day 1 to day 7 (at 1-10 μg/ml) produced the highest yields of CTNT and NKX2.5 positive cardiac population. The percentage yield was greater than 80% without further purification or lactate treatment (FIGS. 25 and 26).

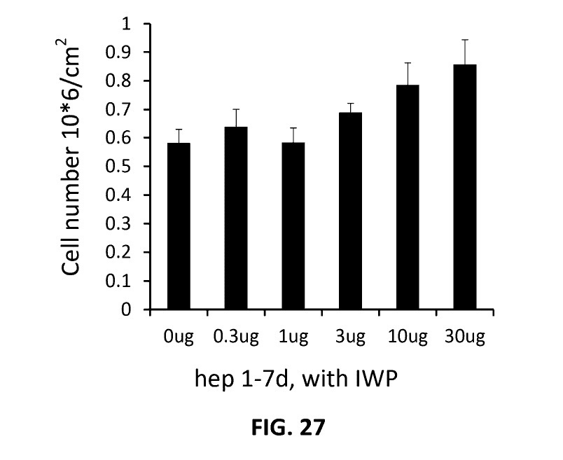

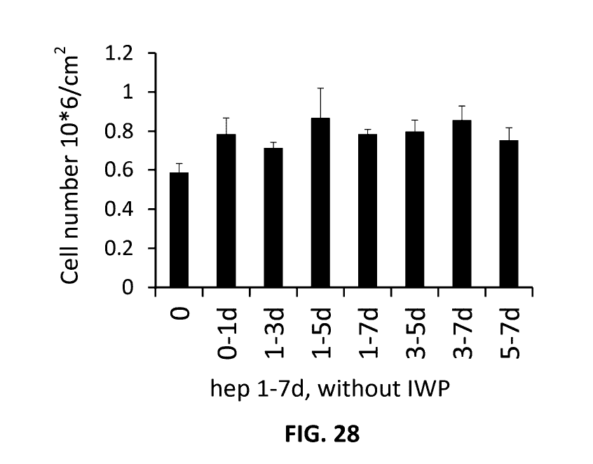

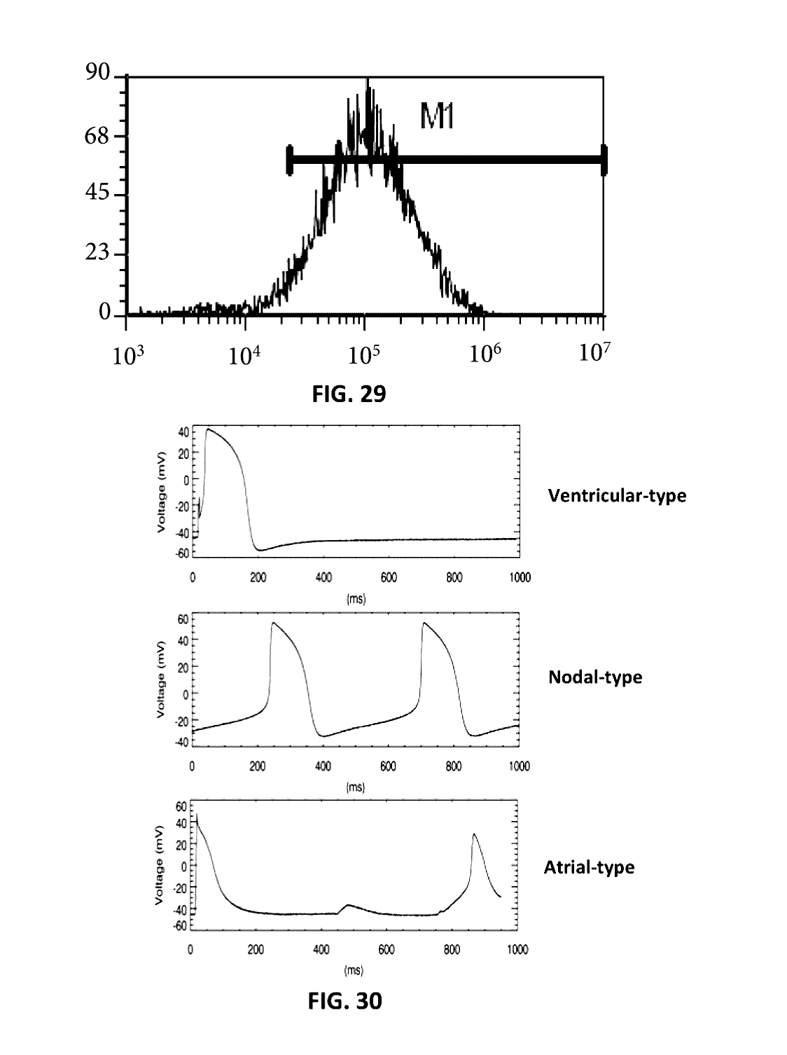

Beginning with a plating density of H1 ESCs of 4×10⁴ cells/cm² on day-2, the total number of live cells derived after 10 days differentiation was about 5-10×10⁵ cells/cm² (and >80% were cardiomyocytes) (FIGS. 27, 28), which increased cell density more than 10-fold. The derived cardiomyocytes were identified by positive immunofluorescence of multiple cardiac markers, and flow cytometry results showed that the population of CTNT positive cells could reach as high as 95% (FIG. 29). Applying patch-clamp analysis, the inventors observed the action potentials of all three cardiomyocyte subtypes (ventricular, nodal, and atrial cardiomyocytes) in the cardiomyocyte population derived with heparin/IWP2 treatment (FIG. 30).

Heparin Alone Promoted Cardiac Differentiation from hPSCs without Treatment with Wnt Inhibitors

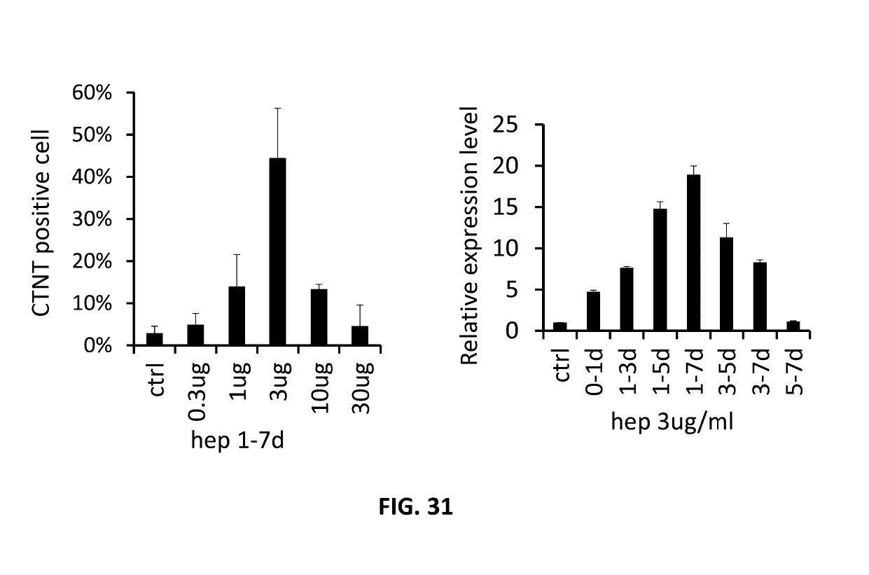

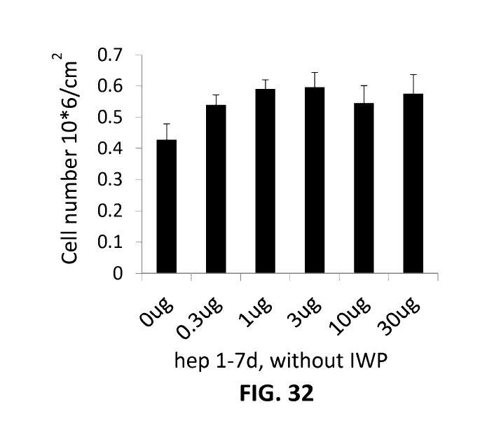

In order to understand how heparin promotes cardiomyocyte differentiation, heparin was applied to hPSCs differentiation at days 1-7 with or without WNT inhibition by IWP2. Surprisingly, heparin treatment alone significantly increased the percentage of NKX2.5 and CTNT positive cells after 10 days differentiation, even though IWP2 and heparin co-treatment produced the highest efficiency of cardiac derivation. The dosage screening suggested that the treatment of 3 μg/ml of heparin on days 1-7 produced the most efficient cardiac derivation rate with comparable cell number yield as IWP2 treatment (FIGS. 31 and 32). The heparin (Sigma) used in the study is an unfractionated heparin sodium salt purified from porcine intestinal mucosa, which may contain other contaminants. To confirm the specific effect of heparin in cardiac differentiation, the inventors co-treated with bacteroides heparinase I and heparin in the differentiation media. Heparinase I is the enzyme that selectively cleaves the glycosidic linkage between hexosamines and uronic acids in heparin. The co-treatment with heparinase I dramatically blocked the effect of heparin in the promotion of cardiac differentiation. This result clarified that heparin, and not undefined contaminant(s), was the key factor in promoting cardiac differentiation. Surprisingly, the cardiac cell differentiation seen with IWP2 treatment was also significantly suppressed by heparinase I, suggesting that any endogenous heparin or heparan sulfate might also play a critical role in cardiac differentiation during the Wnt inhibition process. Furthermore, GO analysis of microarray for RNA samples on both day 6 and day 10 demonstrated that cardiogenesis was promoted by heparin both with and without IWP treatment.

### Heparin Played Biphasic Roles in Modulating Wnt Signaling and Cell Growth During Cardiac Differentiation

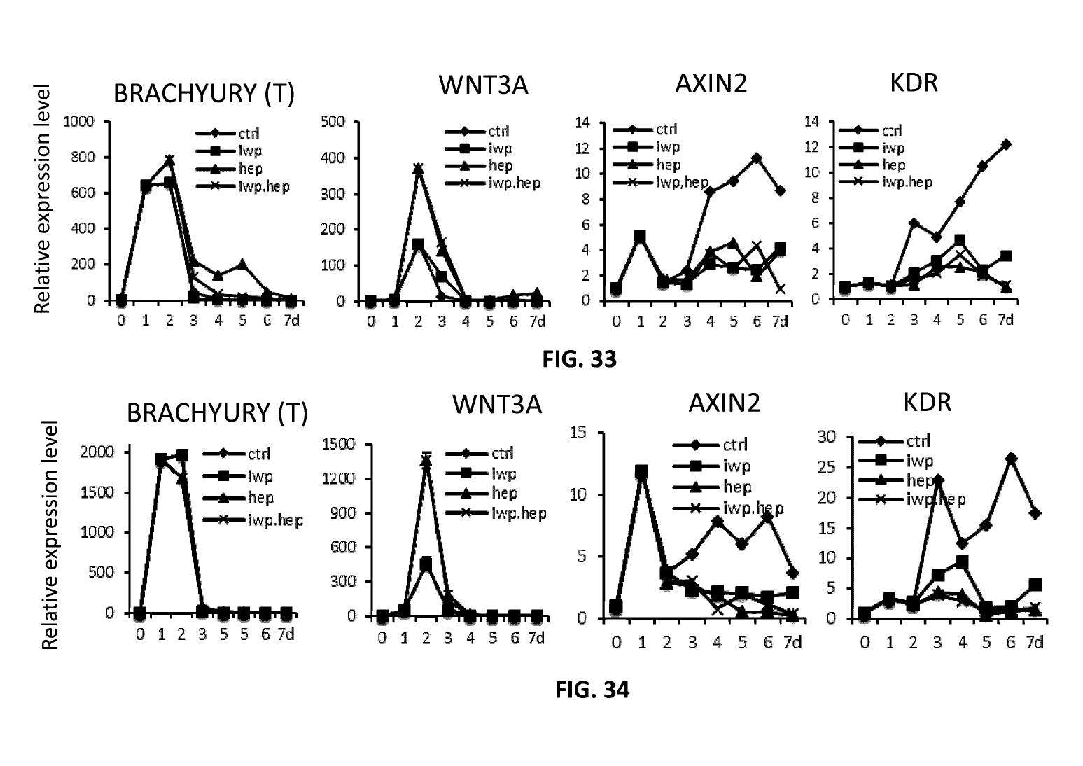

To investigate the function of heparin in mesoderm determination and cardiac specification, stage-specific marker expression levels were measured by qPCR (FIG. 33). The results showed that the expression of early mesoderm marker Brachyury (T) peaked at day 1 and 2 but was not significantly affected when heparin was treated from day 1. However, heparin significantly up-regulated Wnt3A expression at the mesoderm induction stage (days 2-3) while it inhibited Axin2, a downstream target of canonical wnt signaling, at the later cardiac specification stage (days 4-6), indicating that heparin played biphasic roles on Wnt signaling regulation at different stages during cardiac differentiation, which is coincident to the endogenous change of Wnt signaling during mesoderm and cardiac differentiation. Additionally, heparin significantly down-regulated, but did not totally block, the expression of cardiovascular progenitor marker KDR, which has been reported to be expressed at low levels in cardiac-specific mesodermal cells.

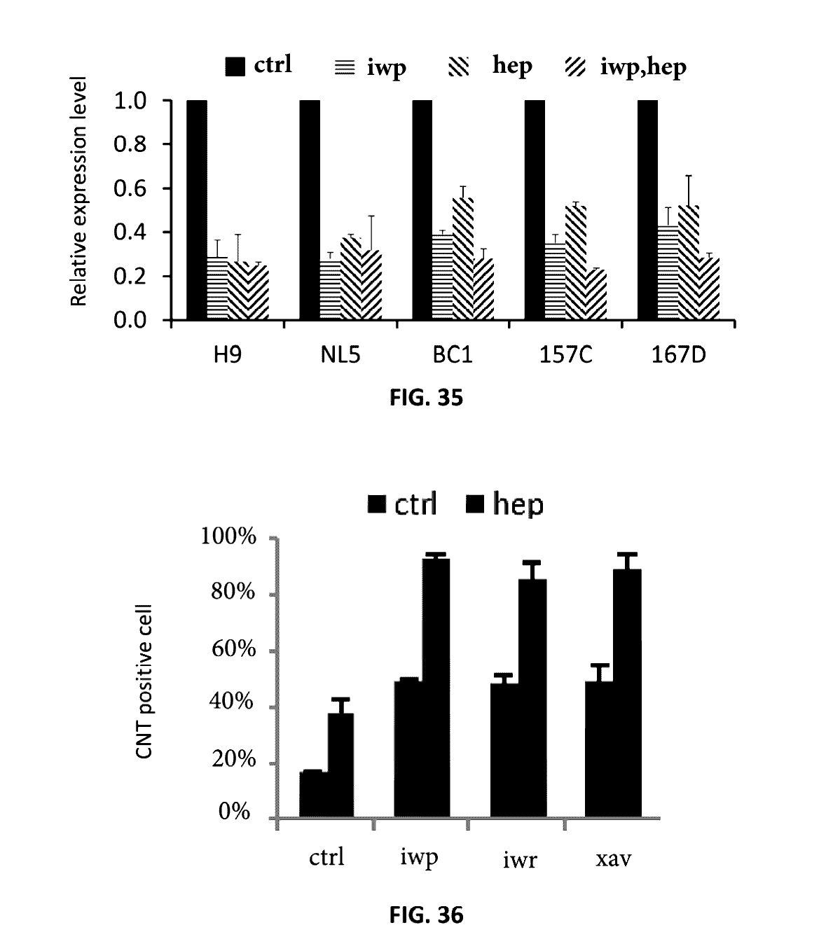

All of these gene expression patterns were reproducible in both human ESC line H1 and iPSC line ND2 (FIG. 34). The inhibition of Axin2 by heparin at day 5 was confirmed in 5 different human PSC lines (FIG. 35). Inhibition of canonical Wnt signaling after day 3 suggests that higher dosages of IWP2 could efficiently produce cardiac differentiation without heparin treatment. But increasing IWP2 dosage did not significantly enhance cardiac differentiation, as shown by CTNT and NKX2.5 staining, which suggested that inhibition of canonical Wnt signaling was not the only pathway affected by heparin.

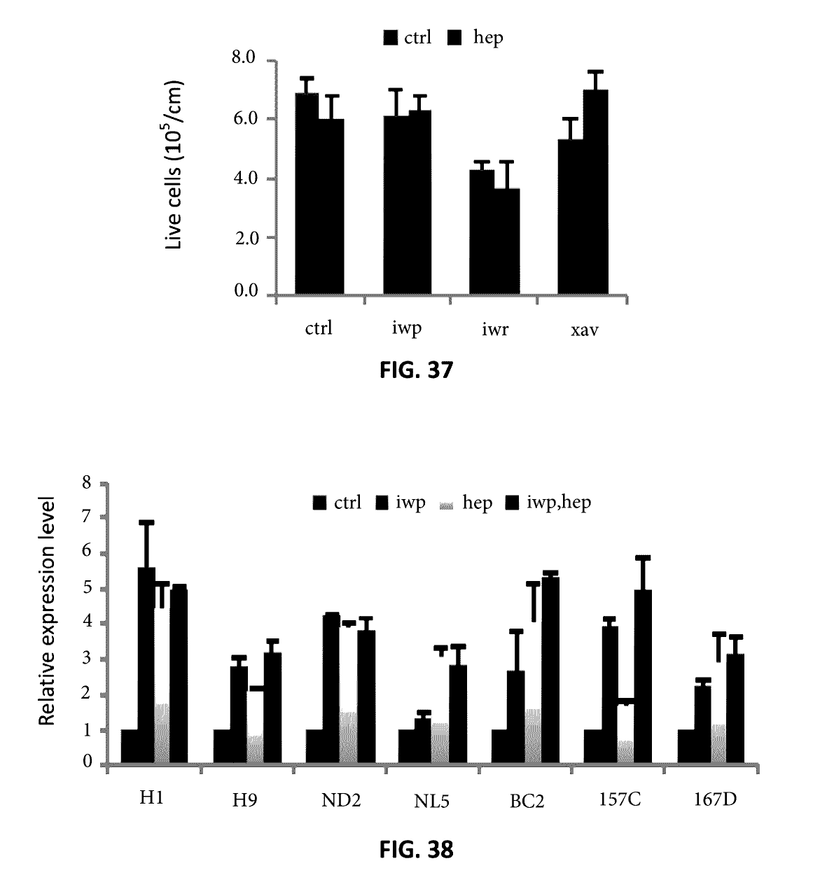

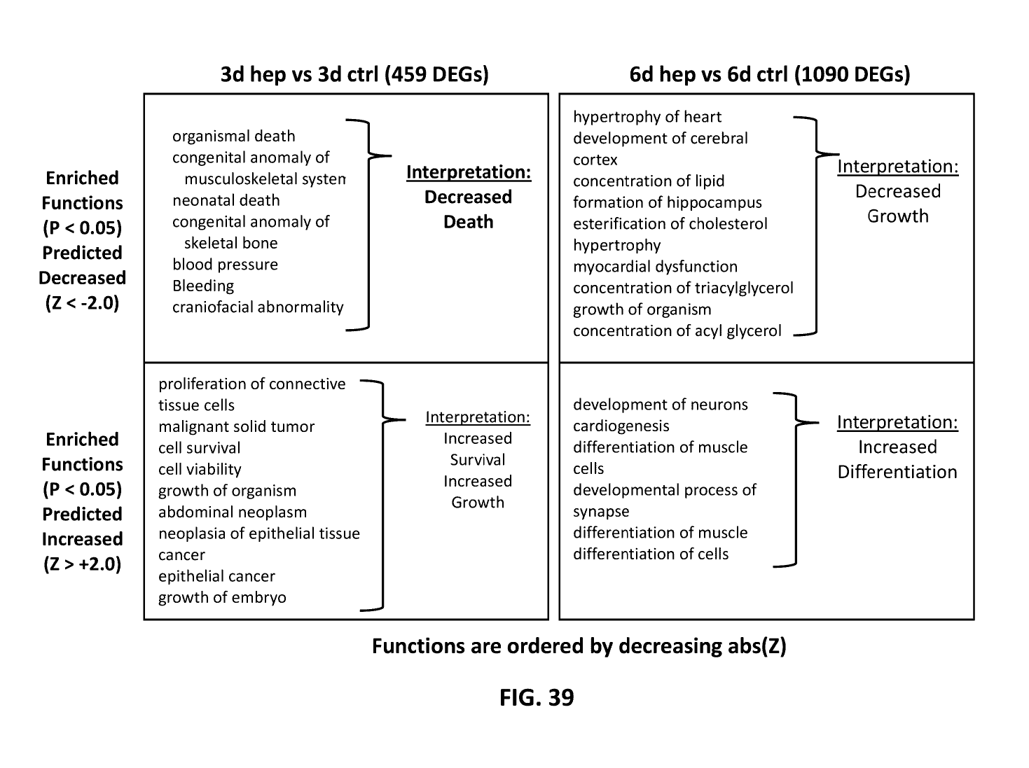

The inventors also evaluated the effects of multiple WNT inhibitors on cardiac differentiation. Results showed that IWP2, IWR1 and XAV939 produced comparable efficiencies of cardiac differentiation in the presence of heparin, but treatment with IWR1 caused more cell death and yielded fewer cardiomyocytes (FIGS. 36, 37). Consistently, the expression of the cardiac progenitor marker, GATA4, was significantly up-regulated by heparin at day 5 as shown in 7 different human PSC lines (FIG. 38). Enriched function prediction by z-score from microarray data comparing heparin treatment with control, showed that heparin played biphasic roles in cell proliferation and differentiation between day 3 and day 6. At day 3, heparin significantly increased cell growth, but decreased cell death, while at day 6, heparin promoted cell differentiation but inhibited cell growth (FIG. 39). In the enriched function predictions, 577 genes were significantly up- or down-regulated by heparin in either day 3 (313 genes) or day 6 (301 genes). Heat mapping indicated that 37 shared genes were regulated in both day 3 and day 6, while heparin produced an opposite regulation on most of these genes between day 3 and day 6.

Cardiomyocyte Differentiation on Defined Matrices from Multiple hPSC Lines

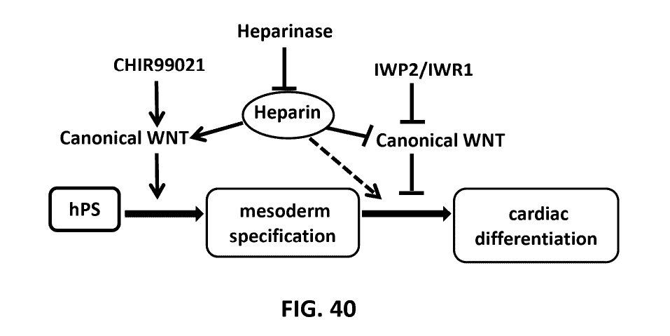

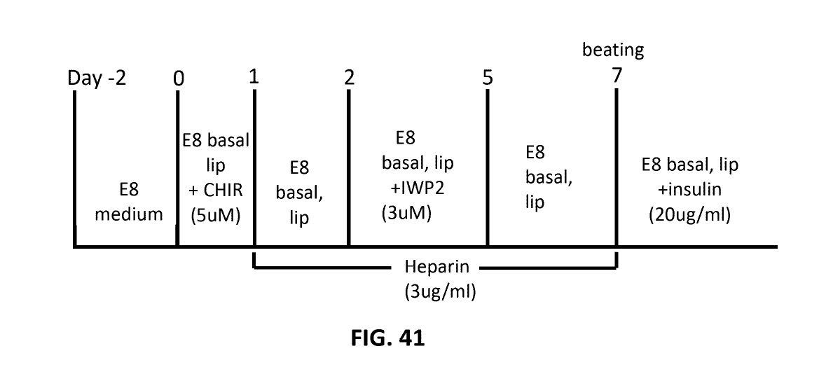

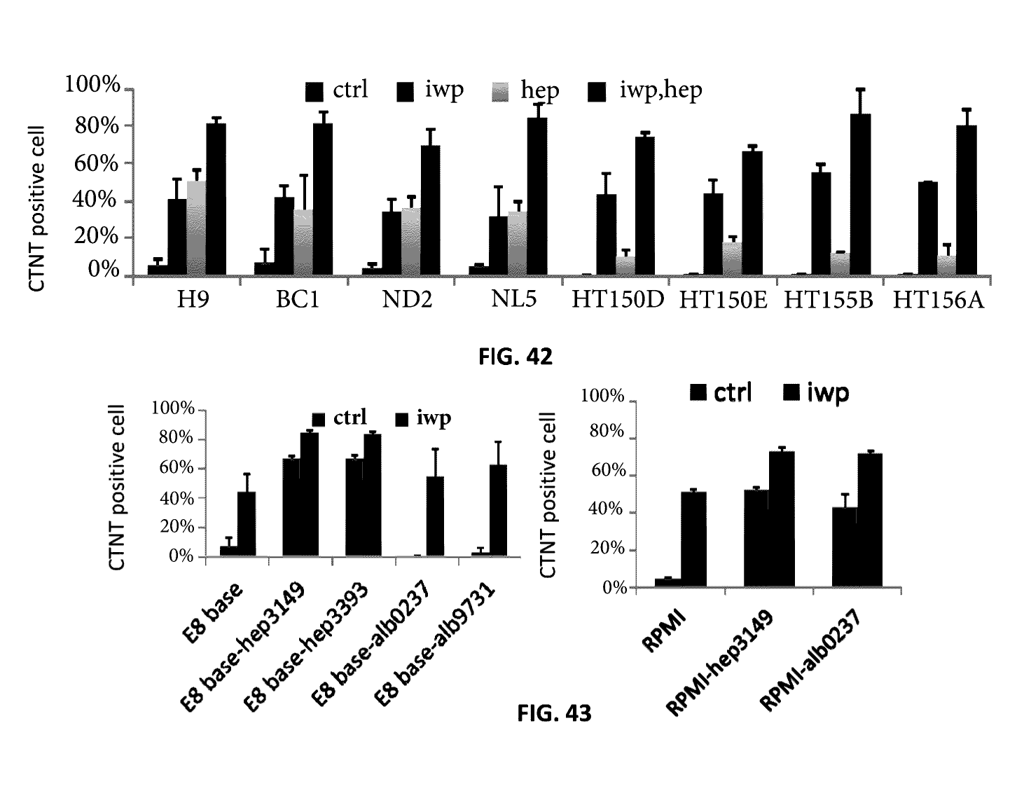

Based on these results, the inventors developed a working model of heparin on cardiomyocyte differentiation in chemically defined conditions (FIG. 40). In this model, heparin has a biphasic effect on regulating canonical Wnt signaling during different stages, and its WNT inhibitory role significantly enhanced cardiac differentiation. Based on this model, the inventors established an efficient cardiac differentiation method based on a commercially-available xeno-free E8 medium platform (diagramed in FIG. 41). In this method, no albumin was used, and the treatment with heparin dramatically enhances cardiac derivation efficiency from 50% to as high as 95%-troponin T positive-cells after 7-10 days differentiation (FIG. 38). In this study, MATRIGEL™ was the main extracellular matrix material used in hPSC maintenance and differentiation since it is the most common matrix used. However, MATRIGEL™ is undefined and may contain animal products. To develop a xeno-free, chemically-defined protocol, the inventors further tested these heparin-based cardiac differentiation methods in defined matrix surfaces, such as xeno-free, human recombinant Vitronectin and Synthemax coated plates. The protocol diagramed in FIG. 41 provided comparable results when conducted on undefined MATRIGEL™ or defined Vitronectin or Synthemax. Additionally, because this protocol was developed on the human H1 ESC line, the inventors also tested H9 ESCs and more than 10 human patient iPSC lines (containing both male and female lines and derived by multiple methods), and the results confirmed that heparin significantly enhanced cardiomyocyte derivation efficiency with or without IWP2 treatment. Using the combination of heparin and IWP2 treatment, all the tested hPSC lines have the capacity to generate 80% or greater troponin T-positive cardiomyocytes after 10 days of differentiation (FIG. 42).

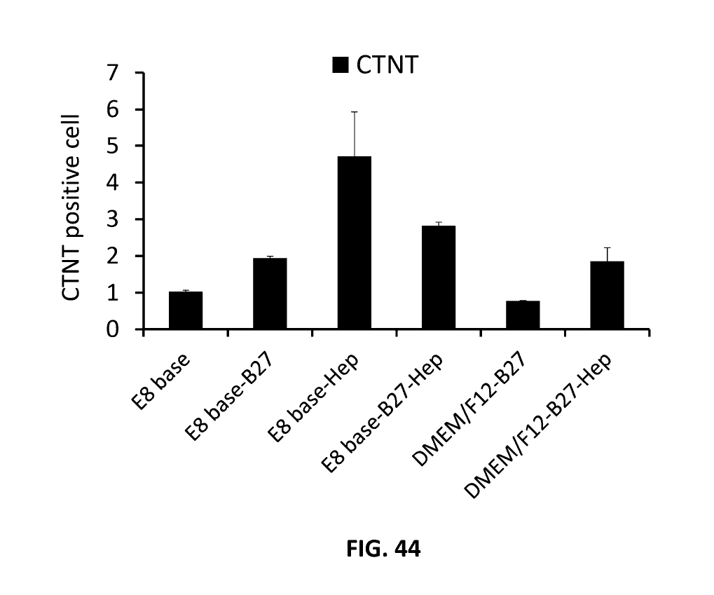

After establishing heparin as an efficient cardiac-promoting reagent, we revisited B27 and albumin's role in cardiac differentiation. Most current protocols in the literature used RPMI basal medium for cardiac differentiation, because DMEM/F12 was reported to be less efficient (Lian, et al., PNAS, 2012, supra; Burridge et al., Cell stem cell, 2012, supra). However, our study demonstrated that E8 basal medium (as defined above) could be used in a high-efficiency cardiac cell differentiation protocol that included heparin treatment. Comparing heparin and albumin effects in these two media (RPMI basal and E8 basal), the inventors found that heparin promoted cardiac differentiation in both basal media with or without IWP2, while the albumin effect was base-media dependent: albumin had a positive impact in RPMI media (with or without IWP2); while in E8 basal media, albumin alone couldn't promote cardiac differentiation and had only a marginal improvement in the presence of IWP2 (FIG. 43). This suggests that heparin and albumin could have different mechanisms of regulating cardiac differentiation, and albumin's effect would therefore be base-medium-dependent. In addition, the inventors also demonstrated that in E8 basal medium or DMEM/F12 media including IWP2, B27 (without insulin) was not as efficient as heparin to promote hPSC cardiac differentiation (FIG. 44).

It was recently reported that albumin had an inhibitory effect on WNT signaling and careful modulation of WNT activation/inhibition alone allowed efficient cardiac differentiation for specific hPSC lines in the absence of albumin (Lian et al., Nature methods 2015, 12(7):595-96). However, such methods might need individualized optimization for different cell lines, because different cells often respond to WNT inhibition differently (FIGS. 35 and 42). Instead, the inventors found that the presence of heparin, along with a WNT inhibitor, promoted efficient cardiac differentiation for all the cell lines tested, and such treatment could help establish a robust and uniform differentiation platform for different cell lines.

To evaluate the potential application of clinical transplantation, the inventors further tested the in vivo survival and incorporation of these iPSC-derived cardiomyocytes in an animal model. Beating cardiomyocytes were derived from EGFP-labeled iPSCs and injected into the myocardium of immunodeficient NSG mice after ligation of the left anterior descending artery. Two or seven-weeks after transplantation, respectively, the mice were sacrificed and the cross-sections of the hearts were stained with antibodies of CTNT and Desmin. EGFP-positive cells still existed in the infarcted area and the fluorescence was overlapped with CTNT and Desmin staining. These results indicate that the iPSC-derived cardiomyocytes could survive and incorporate into infarcted mouse heart, as required for cellular therapy.

### Discussion

These studies used an albumin-free culture media platform to identify heparin as a cardiac-differentiation promoting reagent. Heparan sulfate molecules are proteoglycans that are present in the whole body and serum and expressed endogenously on cell surfaces and in the extracellular matrix. Most biological functions of heparan sulfate are mediated by interactions with proteins that can be promoted or inhibited by exogenous heparin. Heparan sulphate proteoglycans have the ability to regulate the activity of growth factors, act as co-receptor of the growth factor cell surface receptor, control growth factor diffusion through the extracellular matrix, and extend the half-life of growth factors (Rider, Biochemical Society transactions 2006, 34(Pt 3):458-60; Fuerer, et al., Developmental dynamics 2010, 239(1):184-90; Kraushaar, et al., JBC, 2012, 287(27):22691-700). Heparin has been reported to regulate neural development, cardiomyocyte hypertrophy, and heart function, but it has never been linked to cardiomyocyte cell fate determination before these studies. Using heparin, cardiomyocytes may be generated with chemical activation and inhibition of WNT signaling in chemically-defined culture media. This process could be carried out either on an undefined surface (such as MATRIGEL™) or on defined surfaces (such as recombinant Vitronectin or synthetic RGD peptides). It is important to note that like most unfractionated or low molecular weight heparins used in clinical practice and research, the heparins used in these studies were also unfractionated and from porcine sources, which could raise concerns regarding the purity and functional specificity. But the inventors' experiments with the co-treatment of heparinase clarified the specific function of heparin in the promotion of cardiac cell differentiation. The data showing that different batches of heparins from Sigma worked comparably also demonstrates the consistent effects of heparin in this process. Meanwhile, synthetic heparin is now available, such that a fully chemically-defined, animal free production process could be implemented based on the use of synthetic heparin.

Besides the practical value of heparin in cardiac differentiation, this study also revealed the biphasic effect of heparin on WNT signaling as well as on cell proliferation and differentiation at different cardiac differentiation stages. Such effects were not previously reported despite the fact that the interaction between heparin and WNT signaling has been documented and WNT signaling was well known to play biphasic roles in heart development and cardiac development. These findings indicate the complexity of the cell culture platform, because heparin is not only an important serum component, but also a widely used reagent in cell differentiation and in the treatment of cell types, including mesenchymal stem cells, hematopoietic stem cells. Heparin's roles in those studies were often attributed to the stimulation of FGF pathways, while the inhibitory effect of heparinase in differentiation with IWP treatment in the present studies suggested that endogenous polyglycan-dependent regulation plays an essential role in cardiac differentiation (FIG. 2C).

These studies demonstrate that albumin is not an essential factor for hPSC in vitro cardiomyocyte differentiation, and DMEM/F12-based E8 basal medium can produce highly efficient cardiac differentiation in the presence of heparin. The option to eliminate albumin from cell culture media is important for both basic and translational applications. Human recombinant albumin is expensive, and the quality may vary significantly between different batches and suppliers. Additionally, animal components, such as bovine serum albumins, may also lead to additional regulatory burdens when developing clinically compliant materials. Using heparin instead of albumin or B27 in cardiomyocyte derivation may be beneficial in establishing an efficient and cost-effective production process for future large-scale requirements (compare the current costs for making 500 ml of differentiation media when including:

heparin (currently approx. $0.30) versus

human recombinant albumin (currently approx. $15) versus

B27 minus insulin (currently approx. $50).

Several different batches of heparins all displayed a high efficiency promotion of cardiac differentiation. Thus, this advance could eliminate inconsistencies due to source material batch differences that commonly occur in albumin- or B27-based procedures, and significantly reduce the cost of media. In summary, the inventors' cell culture media and procedures provide efficient and cost-effective methods to derive cardiomyocytes from hPSCs in fully chemically-defined conditions.

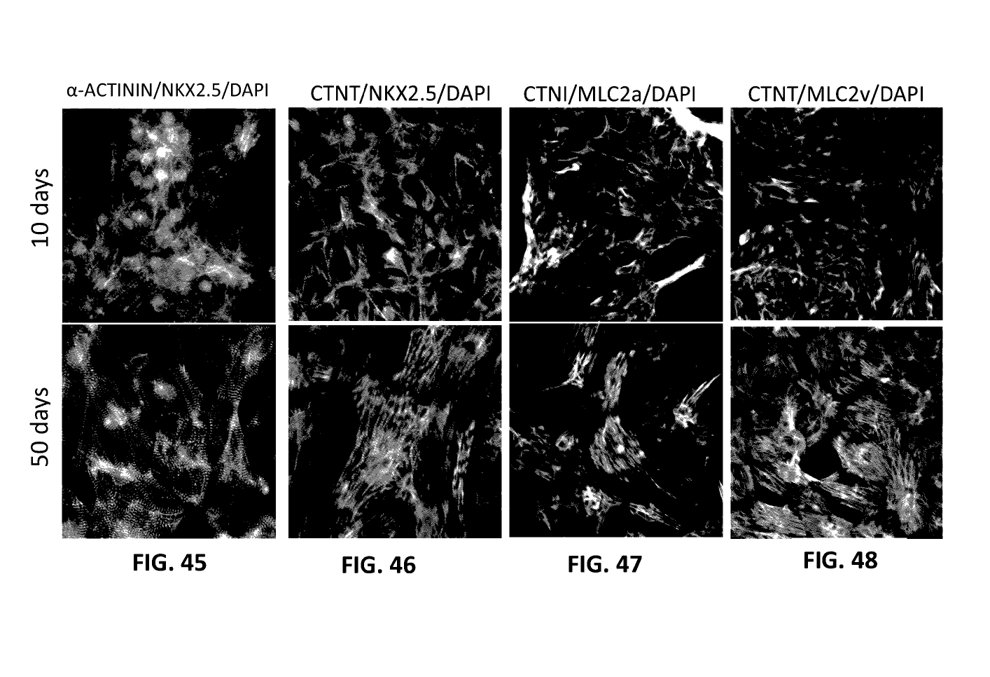

To detect the maturation potential of the derived cardiomyocytes, the inventors cultured the cells to 50 days after induced differentiation. Confocal immunofluorescence of α-ACTININ and CTNT showed significantly increased myofibril content and sarcomere alignment at day 50 compared with day 10 (FIGS. 45 and 46, respectively), suggesting the maturation of these cardiomyocytes at day 50. In these figures, the switch expression of two major isoforms of Myosin Light Chain 2 (MLC2a and MLC2v) has been applied to indicate the maturation of cardiomyocytes. MLC2a is expressed in majority of cardiomyocytes in developmental heart and early stage of cultured cardiomyocytes, while in adult heart and long-term cultured cardiomyocytes, those ventricular cardiomyocytes lost MLC2a expression but express MLC2v specially. MLC2a and MLC2v were co-stained with cardiac troponin on derived cardiomyocytes at 10 days and 50 days after differentiation. At day 10, the majority of cardiomyocytes (CTNI positive; FIG. 47) expressed MLC2a but not MLC2v; while at day 50, the majority of cardiomyocytes (CTNT positive; FIG. 48) expressed MLC2v but not MLC2a. This demonstrates the maturation of these cardiomyocytes after long-term culture.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A cell culture media comprising
a. DMEM/F-12;
b. L-ascorbic acid;
c. selenium;
d. transferrin;
e. at least one lipid selected from the group consisting of: arachidonic acid, cholesterol, linoleic acid, linolenic acid, myristic acid, oleic acid, palmitic acid, and palmitoleic acid; and,
f. at least one compound selected from the group consisting of: CHIR99021, IWP-2, heparin, heparan, insulin, and Rho-associated coiled-coil containing protein kinase (ROCK) inhibitor Y-27632.

2. The culture media of claim 1, wherein the media is an albumin-free medium.

3. The culture media of claim 1, wherein the media contains no insulin.

4. The culture media of claim 1, wherein the media contains no basic fibroblast growth factor (bFGF).

5. The culture media of claim 1, wherein the media contains no transforming growth factor beta (TGFβ).

6. The culture media of claim 1, wherein the at least one lipid comprises arachidonic acid, cholesterol, linoleic acid, linolenic acid, myristic acid, oleic acid, palmitic acid, and palmitoleic acid.

7. The culture media of claim 1, further comprising an antibiotic selected from penicillin, streptomycin, and a combination thereof.

8. The culture media of claim 1, wherein the at least one compound consists of CHIR99021.

9. The culture media of claim 1, wherein the at least one compound consists of heparin.

10. The culture media of claim 1, wherein the at least one compound consists of heparin and IWP-2.

11. The culture media of claim 1, wherein the at least one compound consists of insulin.

12. An in vitro method of directed cardiomyocyte differentiation comprising:
a. culturing a stem cell to a confluence between about 70% and about 90% confluent;
b. contacting the stem cells with a culture media comprising
i. DMEM/F-12;
ii. L-ascorbic acid;
iii. selenium;
iv. transferrin;
v. at least one lipid selected from the group consisting of: arachidonic acid, cholesterol, linoleic acid, linolenic acid, myristic acid, oleic acid, palmitic acid, and palmitoleic acid;
vi. an antibiotic; and,
vii. at least one compound selected from the group consisting of: CHIR99021, IWP-2, heparin, heparan, insulin, and Y-27632;
thereby inducing the differentiation of cardiomyocyte cells.

13. The method of claim 12, wherein the wherein the media contains no albumin, insulin, basic fibroblast growth factor (bFGF), and transforming growth factor beta (TGFβ).

14. The method of claim 12, wherein the at least one compound consists of CHIR99021.

15. The method of claim 12, wherein the at least one compound consists of heparin.

16. The method of claim 12, wherein the at least one compound consists of heparin and IWP-2.

17. The method of claim 12, wherein the at least one compound consists of insulin.

18. The method of claim 12, wherein the culturing comprises culturing a stem cell to a confluence of between 80% to 90% confluent

19. The method of claim 12, wherein the stem cell is at least one of a totipotent, pluripotent, multipotent, oligopotent, or unipotent stem cell, an embryonic stem cell (ESCs), an induced pluripotent stem cell (iPSCs), a fetal stem cell, an adult stem cell, a human stem cell, a human embryonic stem cell, and a human induced pluripotent stem cell.

20. An in vitro method of directed cardiomyocyte differentiation comprising:
a. culturing human induced pluripotent stem cells to a confluence between about 70% and about 90% confluent;
b. thereafter culturing the stem cells for about 24 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; an antibiotic; and CHIR99021;
c. thereafter culturing the stem cells for about 24 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; and an antibiotic;
d. thereafter culturing the stem cells for about 72 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; an antibiotic, heparin, and IWP-2;
e. thereafter culturing the stem cells for about 48 hours in a culture media consisting of DMEM/F-12; L-ascorbic acid; selenium; transferrin; at least one lipid selected from the group consisting of: arachidonic acid, cholesterol, linoleic acid, linolenic acid, myristic acid, oleic acid, palmitic acid, and palmitoleic acid; and an antibiotic;
thereby inducing the differentiation of cardiomyocyte cells.
